SEQUENCE LISTING (10 sequences)

SEQ ID NO: 1 - protein, 338 aa, Artificial Sequence
  LplA-wt wild-type lipoic acid ligase
MSTLRLLISDSYDPWFNLAVEECIFRQMPATQRVLFLWRNADTVVIGRAQNPWKECNTRRMEEDNVRLARRSSGGGAVFHDLGNTCFTFMAGKPEYDKTISTSIVLNALNALGVSAEASGRNDLVVKTTEGDRKVSGSAYRETKDRGFHHGTLLLNADLSRLANYLNPDKKKLAAKGITSVRSRVTNLTELLPEITHEQVCEAIREAFFAHYGERVEAEIISPDKTPDLPNFAETFARQSSWEWNFGQAPAFSHLLDERFTWGGVELHFDVEKGHITRAQVFTDSLNPAPLEALAGRLQGCLYRADMLQQECEALLVDFPEQEKELRELSTWIAGAVR

SEQ ID NO: 2 - protein, 13 aa, Artificial Sequence
  polypeptide
GFEIDKVWYDLDA

SEQ ID NO: 3 - protein, 13 aa, Artificial Sequence
  hypothetical protein
EFDDWKYADVGLI

SEQ ID NO: 4 - protein, 347 aa, Artificial Sequence
  LplA-His6
MKHHHHHHHMSTLRLLISDSYDPWFNLAVEECIFRQMPATQRVLFLWRNADTVVIGRAQNPWKECNTRRMEEDNVRLARRSSGGGAVFHDLGNTCFTFMAGKPEYDKTISTSIVLNALNALGVSAEASGRNDLVVKTVEGDRKVSGSAYRETKDRGFHHGTLLLNADLSRLANYLNPDKKKLAAKGITSVRSRVTNLTELLPGITHEQVCEAITEAFFAHYGERVEAEIISPNKTPDLPNFAETFARQSSWEWNFGQAPAFSHLLDERFTWGGVELHFDVEKGHITRAQVFTDSLNPAPLEALAGRLQGCLYRADMLQQECEALLVDFPEQEKELRELSAWMAGAVR

SEQ ID NO: 5 - protein, 354 aa, Artificial Sequence
  LplA-His6 with TEV cleavage site
MKHHHHHHENLYFQGHMSTLRLLISDSYDPWFNLAVEECIFRQMPATQRVLFLWRNADTVVIGRAQNPWKECNTRRMEEDNVRLARRSSGGGAVFHDLGNTCFTFMAGKPEYDKTISTSIVLNALNALGVSAEASGRNDLVVKTVEGDRKVSGSAYRETKDRGFHHGTLLLNADLSRLANYLNPDKKKLAAKGITSVRSRVTNLTELLPGITHEQVCEAITEAFFAHYGERVEAEIISPNKTPDLPNFAETFARQSSWEWNFGQAPAFSHLLDERFTWGGVELHFDVEKGHITRAQVFTDSLNPAPLEALAGRLQGCLYRADMLQQECEALLVDFPEQEKELRELSAWMAGAVR

SEQ ID NO: 6 - protein, 340 aa, Artificial Sequence
  DHisLplA
GHMSTLRLLISDSYDPWFNLAVEECIFRQMPATQRVLFLWRNADTVVIGRAQNPWKECNTRRMEEDNVRLARRSSGGGAVFHDLGNTCFTFMAGKPEYDKTISTSIVLNALNALGVSAEASGRNDLVVKTVEGDRKVSGSAYRETKDRGFHHGTLLLNADLSRLANYLNPDKKKLAAKGITSVRSRVTNLTELLPGITHEQVCEAITEAFFAHYGERVEAEIISPNKTPDLPNFAETFARQSSWEWNFGQAPAFSHLLDERFTWGGVELHFDVEKGHITRAQVFTDSLNPAPLEALAGRLQGCLYRADMLQQECEALLVDFPEQEKELRELSAWMAGAVR

SEQ ID NO: 7 - protein, 235 aa, Artificial Sequence
  2G10 Fab Light Chain
LFAIPLVVPFYSHSALDVVMTQSPLSLPVTPGEPASISCRSSQSLLRSNGYNYLDWYLQKPGQSPQLLIYLGSIRASGVPDRFSGSGSGTDFTLRISRVEAEDVGVYYCMQALQTPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 255 aa, Artificial Sequence
  2G10 Fab Heavy Chain
MAQVQLQQSGPGLVKPSQTLSLTCAISGDSVSSNSAAWNWIRQSPSRGLEWLGRTYYRSKWYNDYAVSVKSRIIINPDTSKNQFSLQLNSVTPEDTAVYYCARDPGGPLDDSFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAAAHHHHHHGAAEQKLISEEDLNGAA

SEQ ID NO: 9 - protein, 235 aa, Artificial Sequence; identical to SEQ ID NO: 7
  2G10-LAP Fab Light Chain

SEQ ID NO: 10 - protein, 252 aa, Artificial Sequence
  2G10-LAP Fab Heavy Chain
MAQVQLQQSGPGLVKPSQTLSLTCAISGDSVSSNSAAWNWIRQSPSRGLEWLGRTYYRSKWYNDYAVSVKSRIIINPDTSKNQFSLQLNSVTPEDTAVYYCARDPGGPLDDSFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAAHHHHHHAAGFEIDKVWYDLDA

(19) United States
(12) Patent Application Publication
(10) Pub. No.: US 2021/0196842 A1
(43) Pub. Date: Jul. 1, 2021

(54) SITE-SPECIFIC RADIOFLUORINATION OF PEPTIDES WITH 8-[18F]-FLUOROOCTANOIC ACID CATALYZED BY LIPOIC ACID LIGASE

(71) Applicant: THE REGENTS OF THE UNIVERSITY OF CALIFORNIA, Oakland, CA (US)

(72) Inventors: Christopher DRAKE, San Francisco, CA (US); Henry VANBROCKLIN, San Francisco, CA (US); Charles CRAIK, San Francisco, CA (US); Natalia SEVILLANO, San Francisco, CA (US)

(73) Assignee: The Regents of the University of California, Oakland, CA (US)

(21) Appl. No.: 15/777,558

(22) PCT Filed: Nov. 29, 2016
(86) PCT No.: PCT/US2016/063996

§ 371 Date: May 18, 2018

Related U.S. Application Data
(60) Provisional application No. 62/261,015, filed on Nov. 30, 2015.

(51) Int. Cl. A61K 51/08; G01N 33/534

(52) U.S. Cl. CPC A61K 51/088; C12Y 207/07063; G01N 33/534

(57) ABSTRACT

New methodologies for site-specifically radiolabeling proteins with the PET isotope [^{1S}F] are required to generate high quality radiotracers for imaging in both the preclinical and clinical settings. The enzymatic radiofluorination overcomes many of the limitations encountered to date with purely chemical approaches. The bacterial enzyme lipoic acid ligase was used to conjugate [¹⁸F]-fluorooctanoic acid to both a small peptide and a Fab antibody fragment. Labeling was site-specific and highly efficient under mild aqueous conditions using small amounts of peptide/protein (1-10 nmol). The labeled construct retained full epitope binding affinity and was stable in mouse serum. Using an optimized reaction scheme, mCi quantities of [¹⁸F]-Fab were generated, an amount sufficient for human imaging.

### CROSS REFERENCE TO RELATED APPLICATIONS

This application claims the benefit under 35 U.S.C. § 119(e) of U.S. Provisional Application No. 62/261,015 filed on Nov. 30, 2015, which is incorporated herein by reference in its entirety for all purposes.

### BACKGROUND OF THE INVENTION

Positron-emitting tomographic (PET) imaging has found widespread use both in the clinic and in preclinical drug development due to its high sensitivity and ability to generate quantitative data. The functional information from a PET scan can be combined with anatomical information from computed tomography (CT) or magnetic resonance (MR) imaging, giving a powerful tool capable of precisely annotating disease biochemistry in vivo. The PET radionuclide [¹⁸F] exhibits desirable physical properties for imaging, which has led to the development of numerous [¹⁸F]-radiotracers including [¹⁸F]-FDG, [¹⁸F]-choline and [¹⁸F]-FLT. Its intermediate half-life (110.9 mins) requires rapid, highly efficient reaction and purification schemes. While effective protocols have been developed for many small molecules, labeling peptides with higher order structures (e.g. affibodies, diabodies, antibody fragments) with [¹⁸F] remains a significant unmet challenge in the field. The short half-life of [¹⁸F] makes it unsuited for labeling proteins with long in vivo circulation times (e.g., full IgG antibodies). Contemporary screening techniques allow for the evolution of these proteins with highly potent and specific binding to other proteins, nucleic acids and carbohydrates, including numerous targets which cannot be addressed with small molecules. Hence developing methodologies to generate clinically translatable [¹⁸F]-radiotracers from proteins is vital.

The harsh conditions required for C-[¹⁸F] bond formation prohibits direct radiofluorination of unmodified proteins. Several groups have developed small molecule [¹⁸F]-prosthetics which can be coupled to endogenous protein amino acids using mild bioconjugation chemistry (Bioconj. Chem., 2015, 26, 1). The most widely used compound is N-succinimidyl-[¹⁸F]-fluorobenzoate ([¹⁸F]-SFB), an activated ester which reacts with the ε-amine from solvent-exposed lysine residues (Vaidyanathan, et al., Nucl. Med. Biol. 1992, 19(3):275; Vaidyanathan, et al.,Nat. Protoc. 2006, 1(4):1655). However, radiofluorination with [¹⁸F]-SFB has several well recognized limitations, including a lengthy multi-step synthesis for its preparation and coupling to a protein (usually hours), low bioconjugation yields (˜40%) and a lack of control over which lysines are labeled. Reduced immunoreactivities have been reported following lysine modification (Robinson, et al., Cancer Res. 2005, 65(4):1471), presumably due to labeling within the epitope binding region, highlighting the deleterious effect of non-specific labeling. To achieve even the modest conjugation yields reported for [¹⁸F]-SFB, impractically large amounts of protein precursor (>100 nmol) are required, which limits the specific activity of the resulting radiotracer. Alternatives to SFB which target other endogenous amino acids (e.g. [¹⁸F]-FBAM)(Gill, et al., J. Med. Chem. 2009, 52(19):5816; Berndt, et al., Nucl. Med. Biol. 2007, 34:5) or engineered unnatural orthogonally reactive moieties such as oximes or tetrazines (e.g. [¹⁸F]-flurobenzaldehyde, [¹⁸F]-transcyclooctene)(Cheng, et al., J. Nucl. Med. 2008, 49(5):804; Flavell, et al., J. Am. Chem. Soc. 2008, 130(28):9106; Glaser, et al., J. Nucl. Med. 2013, 54(11):1981; Liu, et al., Mol. Imaging 2013, 12(2):121; Rashidian, et al., 2015, 112(19):1) have been developed. Unfortunately, none of these approaches has overcome all of the limitations of [¹⁸F]-SFB and many require prior chemical manipulation of the protein. Direct radiofluorination of proteins pre-modified with [¹⁸F]-acceptor moieties (e.g. NOTA for labeling with Al[¹⁸F]) is an emerging field, however labeling conditions are frequently harsh and proteins must always be chemically manipulated beforehand (Glaser, et al., J. Nucl. Med. 2013, 54(11):1981; Su, et al., Mol. Pharm. 2014, 11:3947).

Many of the aforementioned chemical challenges could be overcome by using an enzyme to conjugate an [¹⁸F]-labeled prosthetic to a protein. Enzymes function optimally in the mild, aqueous conditions required to preserve the integrity of [¹⁸F]-proteins. In addition, enzymatic bioconjugation could facilitate site-specific radiolabeling, a major virtue in contemporary radiotracer development. There are many examples of enzymes which conjugate small molecules to proteins, including farnesyl- and myristoyl- transferases and histone-modifying enzymes. The structural similarities between these post-translational modifications and known [¹⁸F]-prosthetics made us confident that a wild type or modestly engineered enzyme could couple an unnatural [¹⁸F]-prosthetic to a target peptide.

There continues to be a need in the art for site-specifically modified polypeptides that include one or more detectable label. Further, there exists a need to prepare such modified polypeptides quickly with minimal post-labeling purification. This is particularly true for those polypeptides labeled with radioisotopes with short half lives, e.g., ¹⁸F, which is highly valued as a detectable tracer in positron emissing tomography (PET). The art continues to be in need of diagnostic imaging agents of high specific activity. Furthermore, the provision of a class of radiolabeled diagnostic agents of high specific activity, which are substantially homogeneous would represent a significant advance in the field of diagnostic imaging. As set forth hereinbelow, the present invention provides these and other advantages.

### SUMMARY OF THE INVENTION

The invention relates to polypeptide labeling in vivo and in vitro with a detectable label (e.g., a radioisotope, fluorophore, mass spectrometric label). Attempts to label specific polypeptides are often frustrated by a lack of reagents with sufficient specificity. The invention overcomes this lack of specificity through the use of lipoic acid ligase and mutants thereof with lipoic acid analogs and acceptor polypeptides that are recognized by lipoic acid ligase and mutants thereof. The invention includes, in part, use of a lipoic acid ligase to site-specifically and covalently attach small molecules to polypeptides modified by a short peptide tag, which is a recognition sequence for lipoic acid ligase.

Because the method of the invention provides rapid and site-selective labeling of polypeptides with a detectable label, post-labeling purification produces conjugates with a high degree of purity. In addition, the enzymatic nature of the bioconjugation results in high labeling yields using minimal amounts of peptie precursor. This is particularly advantageous when labeling polypeptides for use in PET methods as it is frequently not possible to separate labeled from unlabeled materials. Hence, achieving useful bioconjugation yields with minimal amounts of peptide results in radiotracers with high specific activities, provides a significant benefit for PET imaging. Moreover, the labeling process itself is rapid, resulting in minimal decay of ¹⁸F prior to use of the radiotracer. The combination of these factors leads to radiotracers with high specific activities, that is to say, with a higher ratio of PET-active molecules compared to non-radioactive PET-inactive molecules than might be achieved with other methods. In many situations, this higher ratio of PET-active compounds leads to superior images.

The invention therefore provides, inter alia, methods for labeling proteins in vitro or in vivo. The method generally involves contacting a lipoic acid analog (e.g., a labeled fatty acid) with a fusion protein comprising an acceptor polypeptide in the presence of a wild type lipoic ligase or a mutant of such a ligase, and allowing sufficient time for conjugation of the lipoic acid analog to the fusion protein. Times and reaction conditions suitable for mutant lipoic acid ligase activity will generally be comparable to those for wild-type lipoic acid ligase, which are known in the art.

In various embodiments, the invention provides methods of using bacterial enzyme lipoic acid ligase (Lp1A) for polypeptide radiofluorination. Also provided are radiofluorinated polypeptides and methods of using these polypeptides in diagnostic imaging methodologies, such as positron emission tomography.

Further embodiments, objects and advantages of the invention are set forth in the detailed description that follows.

### BRIEF DESCRIPTION OF THE DRAWINGS

FIG. 1. Schematic Overview of 2G10-Fab-LAP Radiofluorination with [18F]-FA Catalyzed by Lp1A.

FIG. 2A. Schematic overview of conjugation of FA to the LAP peptide catalyzed by Lp1A.

FIG. 2B. RP-HPLC trace demonstrating conversion of LAP peptide to LAP-FA. Identity of LAP-FA confirmed by ESI-MS (m/z=1756.848).

FIG. 3A. RP-HPLC traces demonstrating conjugation of [¹⁸F]-FA to the LAP peptide. [¹⁸F]-FA B. Red-trace: LAP peptide (60 μM), Lp1A (5 μM), [¹⁸F]-FA (˜200 μCi), ATP (3 mM), Mg(OAc)₂ (5 mM); reaction incubated at 37° C. for 10 minutes, quenched via addition of EDTA to a final volume of 180 mM and analyzed by without prior purification by RP-HPLC using Rad-detector.

FIG. 3B. RP-HPLC traces demonstrating conjugation of [¹⁸F]-FA to the LAP peptide. Blue-trace: LAP-FA non-radioactive standard. HPLC eluent was 45:55:0.1 v:v:v MeCN:H₂O:TFA.

FIG. 4A. Analysis of Purified 2G10-Fab-[¹⁸F]LAP A. SEC traces of purified 2G10-Fab-[¹⁸F]-LAP (Red, rad-trace) and 2G10-Fab-LAP (Blue, UV-trace).

FIG. 4B. SDS-PAGE.

FIG. 5. Schematic Overview of Enzymatically Catalyzed Radiofluorination of Protein using Lp1A and [¹⁸F]-FA.

FIG. 6A. Labelling of LAP and scrambled LAP with FA. A: LAP (GFEIDKVWYDLDA, 60 mM), Lp1A (500 nM), FA (750 mM), ATP (3 mM), Mg(OAc)₂ (5 mM).

FIG. 6B. Scrambled LAP (EFDDWKYADVGLI, 60 mM), Lp1A (5 mM), FA (750 mM), ATP (3 mM), Mg(OAc)₂ (5 mM). Aliquots withdrawn at specific time-points and Lp1A activity quenched via addition of EDTA to a final concentration of 180 mM. Samples analyzed by RP-HPLC using a 20 minute gradient of 30-60% MeCN in H₂O+0.1% TFA and UV detection at 220 nm.

FIG. 7. ESI-MS analysis of purified LAP-FA peptide. Analysis by University of Notre Dame Mass spectrometry facility.

FIG. 8A. Typical Radio-TLC Data used to Measure [¹⁸F]-FA Conjugation to LAP Peptide or 2G10-Fab-LAP A. [¹⁸F]-FA.

FIG. 8B. Reaction exhibiting approximately 75% consumption of [¹⁸F]-FA. All radio-TLC developed using 7:3:0.1 EtOAc:hexanes:acetic acid as eluent.

FIG. 9A. Serum Stability of 2G10-Fab-[¹⁸F]-LAP A. Purified 2G10-Fab-[¹⁸F]-LAP.

FIG. 9B. 2G10-Fab-[¹⁸F]-LAP after 1 h incubation in mouse serum at 37° C. HPLC traces measured using BioSep S3000 column eluted at 2 mL/min with aqueous solutions of 100 mM sodium phosphate (pH 6.8) and 300 mM NaCl (2 mL/min).

FIG. 10. Analysis of Binding of 2G10, 2G10-LAP and 2G10-[¹⁹F]-LAP to uPAR Measured using Octet Instrument.

FIG. 11. ¹H NMR of ethyl 8-[[(4-methylphenyl)sulfonyl]oxy]-octanoate (3).

FIG. 12. ¹H NMR of ethyl 8-fluorooctanoate (4).

FIG. 13. ¹H NMR of 8-fluorooctanoic Acid (FA).

FIG. 14A. Polypeptide sequence of LP1A-His6.

FIG. 14B. Polypeptide sequence of LP1A-HIS6 with TEV cleavage site (identified).

FIG. 14C. Polypeptide sequence of DhisLp1A.

FIG. 14D. Polypeptide sequence of 2G10 Fab, Light Chain.

FIG. 14E. Polypeptide sequence of 2G10 Fab, Heavy Chain.

FIG. 14F. 2G10 LAP Fab, Light Chain.

FIG. 14G. 2G10 LAP Fab, Heavy Chain.

### DETAILED DESCRIPTION OF THE INVENTION

### Introduction

Lp1A catalyzes the formation of a stable amide bond between the ε-amine of a lysine residue and a range of structurally distinct alkyl carboxylates (Cohen, et al., Chembiochem 2012, 13(6):888; Cohen, et al., Biochemistry 2011, 50(38):8221; Fernández-Suárez, et al., Nat. Biotechnol. 2007, 25(12):1483; Liu, et al., J. Am. Chem. Soc. 2012, 134(2):792; Uttamapinant, et al., Proc. Natl. Acad. Sci. U.S.A. 2010, 107(24):10914). Due to its substrate plasticity, Lp1A tolerates fatty acid substrate analogs with modest alterations from the natural substrate.

Lp1A exhibits a strong preference for lysines within specific amino acid motifs (Lp1A acceptor peptides), facilitating site-specific labeling of a polypeptide (e.g., radiolabeling). A 13-amino acid target⁻peptide sequence (GFEIDKVWYDLDA, ‘LAP’ peptide) with excellent kinetic labeling properties has been reported and is incorporated into various embodiments of the invention (Puthenveetil, et al., J. Am. Chem. Soc. 2009, 131(45):16430).

Investigators have recognized the virtues of Lp1A biochemistry for other applications in chemical biology, exploiting this enzyme to conjugate a range of functional motifs (e.g. fluorophores, cross-linkers) to LAP-tagged proteins in live cells (Cohen, et al., Chembiochem 2012, 13(6):888; Cohen, et al., Biochemistry 2011, 50(38):8221; Fernández-Suárez, et al., Nat. Biotechnol. 2007, 25(12):1483; Liu, et al., J. Am. Chem. Soc. 2012, 134(2):792; Uttamapinant, et al., Proc. Natl. Acad. Sci. U.S.A. 2010, 107(24):10914; Baruah, et al., Angew. Chemie Int. Ed. 2008, 47:7018; Hauke, et al., Bioconjug. Chem. 2014, 25(9):1632; Slavoff, et al., J. Am. Chem. Soc. 2011, 133(49):19769; Uttamapinant, et al., Angew. Chem. Int. Ed. Engl. 2012, 51(24):5852; Yao, et al., J. Am. Chem. Soc. 2012, 134(8):3720).

Before the invention is described in greater detail, it is to be understood that the invention is not limited to particular embodiments described herein as such embodiments may vary. It is also to be understood that the terminology used herein is for the purpose of describing particular embodiments only, and the terminology is not intended to be limiting. The scope of the invention will be limited only by the appended claims. Unless defined otherwise, all technical and scientific terms used herein have the same meaning as commonly understood by one of ordinary skill in the art to which this invention belongs. Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention. Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number, which, in the context in which it is presented, provides the substantial equivalent of the specifically recited number. All publications, patents, and patent applications cited in this specification are incorporated herein by reference to the same extent as if each individual publication, patent, or patent application were specifically and individually indicated to be incorporated by reference. Furthermore, each cited publication, patent, or patent application is incorporated herein by reference to disclose and describe the subject matter in connection with which the publications are cited. The citation of any publication is for its disclosure prior to the filing date and should not be construed as an admission that the invention described herein is not entitled to antedate such publication by virtue of prior invention. Further, the dates of publication provided might be different from the actual publication dates, which may need to be independently confirmed.

It is noted that the claims may be drafted to exclude any optional element. As such, this statement is intended to serve as antecedent basis for use of such exclusive terminology as “solely,” “only,” and the like in connection with the recitation of claim elements, or use of a “negative” limitation. As will be apparent to those of skill in the art upon reading this disclosure, each of the individual embodiments described and illustrated herein has discrete components and features which may be readily separated from or combined with the features of any of the other several embodiments without departing from the scope or spirit of the invention. Any recited method may be carried out in the order of events recited or in any other order that is logically possible. Although any methods and materials similar or equivalent to those described herein may also be used in the practice or testing of the invention, representative illustrative methods and materials are now described.

In describing the present invention, the following terms will be employed, and are defined as indicated below.

### Definitions

A “peptide” is an oligopeptide, polypeptide, peptide, protein or glycoprotein. The use of the term “peptide” herein includes a peptide having a sugar molecule attached thereto.

“Peptide” refers to a polymer in which the monomers are amino acids and are joined together through amide bonds, alternatively referred to as a peptide. Additionally, unnatural amino acids, for example, homoserine, phenylglycine, aminocyclobutylcarboxylic acid and homoarginine are also included. Amino acids that are not nucleic acid-encoded may also be used in the present invention. Furthermore, amino acids that have been modified to include reactive groups, glycosylation sites, polymers, therapeutic moieties, biomolecules and the like may also be used in the invention. All of the amino acids used in the present invention may be either the D- or L-isomer thereof. The L-isomer is generally preferred. In addition, other peptidomimetics are also useful in the present invention. As used herein, “peptide” refers to both glycosylated and unglycosylated peptides. Also included are peptides that are incompletely glycosylated by a system that expresses the peptide. For a general review, see, Spatola, A. F., in Chemistry and Biochemistry of Amino Acids, Peptides and Proteins, B. Weinstein, eds., Marcel Dekker, New York, p. 267 (1983).

As used herein, “wild type” means the form of the peptide when produced by the cells and/or organisms in which it is found in nature. When the peptide is produced by a plurality of cells and/or organisms, the peptide may have a variety of wild types.

The term “peptide conjugate,” refers to species of the invention in which a peptide is conjugated with a detectable lipoic acid prosthetic as set forth herein.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, □-carboxyglutamate, and O-phosphoserine. Amino acid analogs refers to compounds that have the same basic chemical structure as a naturally occurring amino acid, i.e., an α carbon that is boundlinked to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid.

The present invention also provides for analogs of proteins or peptides which comprise a protein as identified above. Analogs may differ from naturally occurring proteins or peptides by conservative amino acid sequence differences or by modifications which do not affect sequence, or by both. For example, conservative amino acid changes may be made, which although they alter the primary sequence of the protein or peptide, do not normally alter its function. Conservative amino acid substitutions typically include substitutions within the following groups:
glycine, alanine;
valine, isoleucine, leucine;
aspartic acid, glutamic acid;
asparagine, glutamine;
serine, threonine;
lysine, arginine;
phenylalanine, tyrosine.

Modifications (which do not normally alter primary sequence) include in vivo, or in vitro, chemical derivatization of peptides, e.g., acetylation, or carboxylation. Also included are modifications of glycosylation, e.g., those made by modifying the glycosylation patterns of a peptide during its synthesis and processing or in further processing steps; e.g., by exposing the peptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

It will be appreciated, of course, that the peptides may incorporate amino acid residues which are modified and may or may not affect activity. For example, the termini may be derivatized to include blocking groups, i.e. chemical substituents suitable to protect and/or stabilize the N- and C-termini from “undesirable degradation”, a term meant to encompass any type of enzymatic, chemical or biochemical breakdown of the compound at its termini which is likely to affect the function of the compound, i.e. sequential degradation of the compound at a terminal end thereof.

Blocking groups include protecting groups conventionally used in the art of peptide chemistry which will not adversely affect the in vivo activities of the peptide. For example, suitable N-terminal blocking groups can be introduced by alkylation or acylation of the N-terminus. Examples of suitable N-terminal blocking groups include C₁-C₅ branched or unbranched alkyl groups, acyl groups such as formyl and acetyl groups, as well as substituted forms thereof, such as the acetamidomethyl (Acm), Fmoc or Boc groups. Desamino analogs of amino acids are also useful N-terminal blocking groups, and can either be coupled to the N-terminus of the peptide or used in place of the N-terminal reside. Suitable C-terminal blocking groups, in which the carboxyl group of the C-terminus is either incorporated or not, include esters, ketones or amides. Ester or ketone-forming alkyl groups, particularly lower alkyl groups such as methyl, ethyl and propyl, and amide-forming amino groups such as primary amines (—NH₂), and mono- and di-alkylamino groups such as methylamino, ethylamino, dimethylamino, diethylamino, methylethylamino and the like are examples of C-terminal blocking groups. Decarboxylated amino acid analogues such as agmatine are also useful C-terminal blocking groups and can be either coupled to the peptide's C-terminal residue or used in place of it. Further, it will be appreciated that the free amino and carboxyl groups at the termini can be removed altogether from the peptide to yield deamino and decarboxylated forms thereof without affect on peptide activity.

Other modifications can also be incorporated without adversely affecting the activity and these include, but are not limited to, substitution of one or more of the amino acids in the natural L-isomeric form with amino acids in the D-isomeric form. Thus, the peptide may include one or more D-amino acid resides, or may comprise amino acids which are all in the D-form. Retro-inverso forms of peptides in accordance with the present invention are also contemplated, for example, inverted peptides in which all amino acids are substituted with D-amino acid forms.

Acid addition salts of the present invention are also contemplated as functional equivalents. Thus, a peptide in accordance with the present invention treated with an inorganic acid such as hydrochloric, hydrobromic, sulfuric, nitric, phosphoric, and the like, or an organic acid such as an acetic, propionic, glycolic, pyruvic, oxalic, malic, malonic, succinic, maleic, fumaric, tataric, citric, benzoic, cinnamic, mandelic, methanesulfonic, ethanesulfonic, p-toluenesulfonic, salicyclic and the like, to provide a water soluble salt of the peptide is suitable for use in the invention.

Also included are peptides which have been modified using ordinary molecular biological techniques so as to improve their resistance to proteolytic degradation or to optimize solubility properties or to render them more suitable as a therapeutic agent. Analogs of such peptides include those containing residues other than naturally occurring L-amino acids, e.g., D-amino acids or non-naturally occurring synthetic amino acids. The peptides of the invention are not limited to products of any of the specific exemplary processes listed herein.

The terms “targeting peptide” and “targeting agent”, as used herein, refer to species that will selectively localize in a particular tissue or region of the body. The localization is mediated by specific recognition of molecular determinants, molecular size of the targeting agent or conjugate, ionic interactions, hydrophobic interactions and the like. Other mechanisms of targeting an agent to a particular tissue or region are known to those of skill in the art. An exemplary targeting peptide is a MAb or a fragment thereof.

As used herein, “therapeutic moiety” means any agent useful for therapy including, but not limited to, antibiotics, anti-inflammatory agents, anti-tumor drugs, cytotoxins, and radioactive agents. “Therapeutic moiety” includes prodrugs of bioactive agents, constructs in which more than one therapeutic moiety is linked to a carrier, e.g., multivalent agents. Therapeutic moiety also includes peptides, and constructs that include peptides. Exemplary peptides include those disclosed herein. “Therapeutic moiety” thus means any agent useful for therapy including, but not limited to, antibiotics, anti-inflammatory agents, anti-tumor drugs, cytotoxins, and radioactive agents. “Therapeutic moiety” includes prodrugs of bioactive agents, constructs in which more than one therapeutic moiety is linked to a carrier, e.g., multivalent agents.

As used herein, “anti-tumor drug” means any agent useful to combat cancer including, but not limited to, cytotoxins and agents such as antimetabolites, alkylating agents, anthracyclines, antibiotics, antimitotic agents, procarbazine, hydroxyurea, asparaginase, corticosteroids, interferons and radioactive agents. Also encompassed within the scope of the term “anti-tumor drug,” are conjugates of peptides with anti-tumor activity, e.g. TNF-a. Conjugates include, but are not limited to those formed between a therapeutic protein and a lipoic acid prosthetic.

As used herein, “a cytotoxin or cytotoxic agent” means any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracinedione, mitoxantrone, mithramycin, actinomycin D, 1-dehydrotestosterone, glucocorticoids, procaine, tetracaine, lidocaine, propranolol, and puromycin and analogs or homologs thereof. Other toxins include, for example, ricin, CC-1065 and analogues, the duocarmycins. Still other toxins include diphtheria toxin, and snake venom (e.g., cobra venom).

As used herein, “a radioactive agent” includes any radioisotope that is effective in diagnosing or destroying a tumor or other diseased tissue. Examples include, but are not limited to, fluorine-18, zirconium-89, Iodine-123, iodine-124, iodine-125, iodine-131, indium-111, yttrium-90, leutecium-177 and technetium-99m. Additionally, naturally occurring radioactive elements such as uranium, radium, and thorium, which typically represent mixtures of radioisotopes, are suitable examples of a radioactive agent. The metal ions are typically chelated with an organic chelating moiety.

Many useful chelating groups, crown ethers, cryptands and the like are known in the art and can be incorporated into the compounds of the invention (e.g. EDTA, DTPA, DOTA, NTA, HDTA, etc. and their phosphonate analogs such as DTPP, EDTP, HDTP, NTP, etc). See, for example, Pitt et al., “The Design of Chelating Agents for the Treatment of Iron Overload,” In, INORGANIC CHEMISTRY IN BIOLOGY AND MEDICINE; Martell, Ed.; American Chemical Society, Washington, D.C., 1980, pp. 279-312; Lindoy, THE CHEMISTRY OF MACROCYCLIC LIGAND COMPLEXES; Cambridge University Press, Cambridge, 1989; Dugas, BIOORGANIC CHEMISTRY; Springer-Verlag, New York, 1989, and references contained therein.

Additionally, a manifold of routes allowing the attachment of chelating agents, crown ethers and cyclodextrins to other molecules is available to those of skill in the art. See, for example, Meares et al., “Properties of In Vivo Chelate-Tagged Proteins and Polypeptides.” In, MODIFICATION OF PROTEINS: FOOD, NUTRITIONAL, AND PHARMACOLOGICAL ASPECTS;” Feeney, et al., Eds., American Chemical Society, Washington, D.C., 1982, pp. 370-387; Kasina et al., Bioconjugate Chem., 9: 108-117 (1998); Song et al., Bioconjugate Chem., 8: 249-255 (1997).

As used herein, “pharmaceutically acceptable carrier” includes any material, which when combined with the conjugate retains the activity of the conjugate activity and is non-reactive with the subject's immune system. Examples include, but are not limited to, any of the standard pharmaceutical carriers such as a phosphate buffered saline solution, water, emulsions such as oil/water emulsion, and various types of wetting agents. Other carriers may also include sterile solutions, tablets including coated tablets and capsules. Typically such carriers contain excipients such as starch, milk, sugar, certain types of clay, gelatin, stearic acid or salts thereof, magnesium or calcium stearate, talc, vegetable fats or oils, gums, glycols, or other known excipients. Such carriers may also include flavor and color additives or other ingredients. Compositions comprising such carriers are formulated by well known conventional methods.

As used herein, “administering” means oral administration, administration as a suppository, topical contact, intravenous, intraperitoneal, intramuscular, intralesional, intranasal, intracranial, intra-cerebrospinal, or subcutaneous administration, intrathecal administration, or the implantation of a slow-release device e.g., a mini-osmotic pump, to the subject.

The term “isolated” refers to a material that is substantially or essentially free from components, which are used to produce the material. For peptide conjugates of the invention, the term “isolated” refers to material that is substantially or essentially free from components, which normally accompany the material in the mixture used to prepare the peptide conjugate. “Isolated” and “pure” are used interchangeably. Typically, isolated peptide conjugates of the invention have a level of purity preferably expressed as a range. The lower end of the range of purity for the peptide conjugates is about 60%, about 70% or about 80% and the upper end of the range of purity is about 70%, about 80%, about 90% or more than about 90%.

When the peptide conjugates are more than about 90% pure, their purities are also preferably expressed as a range. The lower end of the range of purity is about 90%, about 92%, about 94%, about 96% or about 98%. The upper end of the range of purity is about 92%, about 94%, about 96%, about 98% or about 100% purity.

Purity is determined by any art-recognized method of analysis (e.g., band intensity on a silver stained gel, polyacrylamide gel electrophoresis, HPLC, or a similar means).

“Essentially each member of the population,” as used herein, describes a characteristic of a population of peptide conjugates of the invention in which a selected percentage of the lipoid acid prosthetic is added to a peptide at to multiple acceptor sites on the peptide. “Essentially each member of the population” speaks to the “homogeneity” of the sites on the peptide conjugated to a lipoic acid prosthetic and refers to conjugates of the invention, which are at least about 80%, preferably at least about 90% and more preferably at least about 95% homogenous.

“Homogeneity,” refers to the structural consistency across a population of acceptor moieties to which the lipoic acid prosthetic is conjugated. Thus, in a peptide conjugate of the invention in which each prosthetic is conjugated to an acceptor site having the same structure as the acceptor site to which every other aesthetic is conjugated, the peptide conjugate is said to be about 100% homogeneous. Homogeneity is typically expressed as a range. The lower end of the range of homogeneity for the peptide conjugates is about 60%, about 70% or about 80% and the upper end of the range of purity is about 70%, about 80%, about 90% or more than about 90%.

When the peptide conjugates are more than or equal to about 90% homogeneous, their homogeneity is also preferably expressed as a range. The lower end of the range of homogeneity is about 90%, about 92%, about 94%, about 96% or about 98%. The upper end of the range of purity is about 92%, about 94%, about 96%, about 98% or about 100% homogeneity. The homogeneity of the peptide conjugates is typically determined by one or more methods known to those of skill in the art, e.g., liquid chromatography-mass spectrometry (LC-MS), matrix assisted laser desorption time of flight mass spectrometry (MALDI-TOF), capillary electrophoresis, and the like.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. Alkyl groups which are limited to hydrocarbon groups are termed “homoalkyl”.

The term “alkylene” by itself or as part of another substituent means a divalent radical derived from an alkyl, as exemplified, but not limited, by —CH₂CH₂CH₂CH₂—. Likewise, the term “heteroalkylene” means a divalent radical derived from an heteroalkyl. Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, P, Si and S, and wherein the nitrogen and sulfur atoms may optionally be oxidized and the nitrogen heteroatom may optionally be quaternized. The heteroatom(s) O, N, P and S and Si may be placed at any interior position of the heteroalkyl group or at the position at which the alkyl group is attached to the remainder of the molecule. Examples include, but are not limited to, —CH₂—CH₂13 O—CH₃, —CH₂—CH₂—NH—CH₃, —CH₂—CH₂—N(CH₃)—CH₃, —CH₂—S—CH₂—CH₃, —CH₂—CH₂,—S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, O—CH₃, —O—CH₂—CH₃, and —CN. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. The terms “cycloalkylene” and “heterocycloalkylene,” by themselves or as part of another substituent, means a divalent radical derived from a cycloalkyl or heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “aryl” means, unless otherwise stated, a polyunsaturated, aromatic, hydrocarbon substituent which can be a single ring or multiple rings (preferably from 1 to 3 rings) which are fused together or linked covalently. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. The terms “arylene” and “heteroarylene,” by themselves or as part of another substituent, means a divalent radical derived from a aryl or heteroaryl, respectively.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

The term “oxo” as used herein means an oxygen that is double bonded to a carbon atom.

Each of the above terms (e.g., “alkyl,” “heteroalkyl,” “aryl” and “heteroaryl”) are meant to include both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—OR′, —NR′R″, —SR′, -halogen, —SiR′R″R″′, —OC(O)R′, —C(O)R′, —CO₂R′, —CONR′R″, —OC(O)NR′R″, —NR″C(O)R′, —NR′—C(O)NR″R″′, —NR″C(O)₂R′, —NR—C(NR′R″R″)═NR′″, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R″′ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: halogen, —OR′, ═O, ═NR′, ═N—OR′, —NR′R″, —SR′, -halogen, —SiR′R″R″′, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R″′ and R″″ are preferably independently selected from hydrogen, alkyl, heteroalkyl, aryl and heteroaryl. When a compound of the invention includes more than one R group, for example, each of the R groups is independently selected as are each R′, R″, R″′ and R″″ groups when more than one of these groups is present.

Two of the substituents on adjacent atoms of the aryl or heteroaryl ring may optionally be replaced with a substituent of the formula -T-C(O)—(CRR′)_{q}—U—, wherein T and U are independently —NR—, —O—, —CRR¹—or a single bond, and q is an integer of from 0 to 3. Alternatively, two of the substituents on adjacent atoms of the aryl or heteroaryl ring may optionally be replaced with a substituent of the formula -A-(CH₂)ᵣ-B-, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula—(CRR′)₅—X—(C″R″′)_{d}—, where s and d are independently integers of from 0 to 3, and X′ is —O—, —NR′—, —S—, —S(O)—, —S(O)₂—, or —S(O)₂NR′—. The substituents R, R′, R″ and R″′ are preferably independently selected from hydrogen or substituted or unsubstituted (C₁-C₆)alkyl.

As used herein, the term “heteroatom” is meant to include oxygen (O), nitrogen (N), sulfur (S), phosphorus (P), and silicon (Si).

The neutral forms of the compounds are preferably regenerated by contacting the salt with a base or acid and isolating the parent compound in the conventional manner. The parent form of the compound differs from the various salt forms in certain physical properties, such as solubility in polar solvents.

A “lipoic acid prosthetic” is an organic molecule, which is a substrate for Lp1A and can be added by Lp1A to the acceptor polypeptide, preferably when this polypeptide is fused with a target protein. The “prosthetic” further includes one or more detectable label and/or therapeutic agent. Exemplary prosthetics of the invention further include a reactive functional group providing a locus for conjugation of additional species to the prosthetic either before or after it is conjugated to the polypeptide. An exemplary prosthetic is of a structure according to Formula I:

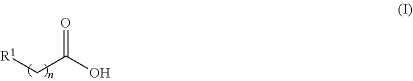

in which R¹ is a reactive functional group, a detectable label, e.g., a radioisotope, a fluorophore or a mass spectrometric label, or R¹ is a therapeutic agent, e.g., a toxin. An exemplary R¹ moiety is ¹⁸F. Alternatively, R¹ is substituted or unsubstituted alkyl, substituted or unsubstituted heteroalkyl, substituted or unsubstituted aryl, or substituted or unsubstituted heteroaryl, functionalized with a detectable moiety or a therapeutic moiety. The index n is an integer from 6 to 18, e.g., from 7 to 15, or from 8 to 13. The methylene moieties are optionally substituted alkyl moieties as described herein.

A lipoic acid prosthetic is a molecule that may be structurally similar to lipoic acid. Exemplary prosthetics include: i) a primary carboxylic acid; ii) an alkyl chain of greater than about 6 substituted or unsubstituted methylene units (e.g., at least 6, 7, 8, 9, 10, 11, 12, 13, 14, 15 or at least 16 substituted or unsubstituted methylene units); and iii) a functional moiety at the omega-position. Lipoic acid prosthetics may share one or more particular structural feature in common with lipoic acid. A lipoic acid prosthetic may be synthesized from lipoic acid, but is not so limited. Examples of lipoic acid prosthetics include, but are not limited to, a fluoroalkyl, fluoroaryl (e.g., a fluoroalkyl or fluoroaryl in which at least one F moiety is an ¹⁸F moiety), an alkyl azide, an alkyne carboxylic acid, an aryl azide photoaffinity probe, a fluorophore (coumarin) substrate, a modified alkyl azide, a modified alkyne, a carboxylic acid, a 4-azido-2,3,5,6-tetrafluorobenzoic derivative, a 7,7′-azo-octanoic acid, a benzophenone, or a 6,8-difluoro-7-hydroxycoumarin fluorophore derivative.

A “detectable label” as used herein is a molecule or compound that can be detected by a variety of methods including fluorescence, electrical conductivity, radioactivity, size, and the like. The label may be of a chemical (e.g., carbohydrate, lipid, etc.), peptide or nucleic acid nature although it is not so limited. The label may be directly or indirectly detectable. The label can be detected directly for example by its ability to emit and/or absorb light of a particular wavelength. A label can be detected indirectly by its ability to bind, recruit and, in some cases, cleave (or be cleaved by) another compound, thereby emitting or absorbing energy. An example of indirect detection is the use of an enzyme label that cleaves a substrate into visible products.

The type of label used will depend on a variety of factors, such as but not limited to the nature of the protein ultimately being labeled. The label should be sterically and chemically compatible with the lipoic acid analog, the acceptor peptide and the target protein. In most instances, the label should not interfere with the activity of the target protein.

Generally, the label can be selected from the group consisting of a fluorescent molecule, a chemiluminescent molecule (e.g., chemiluminescent substrates), a phosphorescent molecule, a radioisotope, an enzyme, an enzyme substrate, an affinity molecule, a ligand, an antigen, a hapten, an antibody, an antibody fragment, a chromogenic substrate, a contrast agent, an MRI contrast agent, a PET label (i.e., a radioisotope), a phosphorescent label, and the like.

Specific examples of labels include radioactive isotopes such as fluorine-18 (˜110 min), carbon-11 (˜20 min), nitrogen-13 (˜10 min), oxygen-15 (˜2 min), gallium-68 (˜67 min), zirconium-89 (˜78.41 hours), or rubidium-82 (˜1.27 min). The times represent half-lives. ³²P or ³H are also of use. Further labels include haptens such as digoxigenin and dintrophenyl; affinity tags such as a FLAG tag, an HA tag, a histidine tag, a GST tag; enzyme tags such as alkaline phosphatase, horseradish peroxidase, beta-galactosidase, etc. Other labels include fluorophores such as fluorescein isothiocyanate (“FITC”), Texas Red®, tetramethylrhodamine isothiocyanate (“TRITC”), 4,4-difluoro-4-bora-3a, and 4a-diaza-s-indacene (“BODIPY”), Cy-3, Cy-5, Cy-7, Cy-Chrome™, R-phycoerythrin (R-PE), PerCP, allophycocyanin (APC), PharRed™, Mauna Blue, Alexa™ 350 and other Alexa™ dyes, and Cascade Blue®.

As used herein, an “acceptor peptide” is a protein or peptide having an amino acid sequence that is a substrate for a lipoic acid ligase, lipoic acid ligase, or mutant thereof, a lipoic acid ligase homolog or mutant thereof (i.e., a lipoic acid ligase homolog or mutant recognizes and is capable of conjugating a lipoic acid analog (“prosthetic”) or lipoic acid to the peptide).

The term “specific activity” is the amount of radioactivity of a radioisotope or radiolabeled compound associated with the physical mass of the element or compound. The accepted units for specific activity are Becquerel (Bq) per gram (Bq/g) or curie (Ci) per gram (Ci/g) or Bq per mole (Bq/mol) or Ci per mole (Ci/mol).

Certain compounds of the invention include one or more “reactive functional group”. Exemplary species include a reactive functional group attached directly to the prosthetic or to a linker attached to the prosthetic. An exemplary reactive functional group is attached to an alkyl or heteroalkyl linker on the prosthetic. When the reactive group is attached a substituted or unsubstituted alkyl or substituted or unsubstituted heteroalkyl linker moiety, the reactive group is preferably located at a terminal position of the alkyl or heteroalkyl chain. Reactive groups and classes of reactions useful in practicing the present invention are generally those that are well known in the art of bioconjugate chemistry. Currently favored classes of reactions available with the prosthetic compounds and polypeptide conjugates of the invention are those proceeding under relatively mild conditions. These include, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). The conditions are sufficiently mild that the prosthetic, the polypeptide and the prosthetic-polypeptide conjugate do not undergo significant degradation under the reaction conditions used to deploy the reactive functional group in a conjugation reaction. Useful reactions are discussed in, for example, March, Advance Organic Chemistry, 3rd Ed., John Wiley & Sons, New York, 1985; Hermanson, Bioconjugate Techniques, Academic Press, San Diego, 1996; and Feeney et al., Modification of Proteins; Advances in Chemistry Series, Vol. 198, American Chemical Society, Washington, D.C., 1982.

Useful reactive functional groups include, for example:
(a) carboxyl groups and derivatives thereof including, but not limited to activated esters, e.g.,N-hydroxysuccinimide esters, N-hydroxyphthalimide, N-hydroxybenztriazole esters, p-nitrophenyl esters; acid halides; acyl imidazoles; thioesters; alkyl, alkenyl, alkynyl and aromatic esters; and activating groups used in peptide synthesis;
(b) hydroxyl groups and hydroxylamines, which can be converted to esters, sulfonates, phosphoramidates, ethers, aldehydes, etc.
(c) haloalkyl groups, wherein the halide can be displaced with a nucleophilic group such as, for example, an amine, a carboxylate anion, thiol anion, carbanion, or an alkoxide ion, thereby resulting in the covalent attachment of a new group at the site of the halogen atom;
(d) dienophile groups, which are capable of participating in Diels-Alder reactions such as, for example, maleimido groups;
(e) aldehyde or ketone groups, allowing derivatization via formation of carbonyl derivatives, e.g., imines, hydrazones, semicarbazones or oximes, or via such mechanisms as Grignard addition or alkyllithium addition;
(f) sulfonyl halide groups for reaction with amines, for example, to form sulfonamides;
(g) thiol groups, which can be converted to disulfides or reacted with acyl halides, for example;
(h) amine, hydrazine or sulfhydryl groups, which can be, for example, acylated, alkylated or oxidized;
(i) alkenes, which can undergo, for example, cycloadditions, acylation, Michael addition, etc;
(j) epoxides, which can react with, for example, amines and hydroxyl compounds; and
(k) phosphoramidites and other standard functional groups useful in nucleic acid synthesis.

In various embodiments, the reactive functional group is a member selected from:

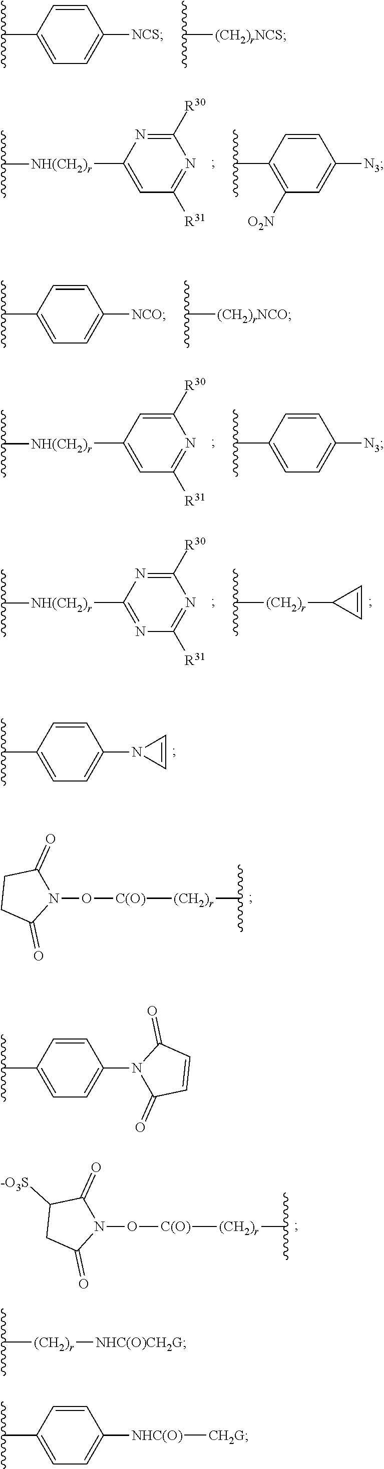

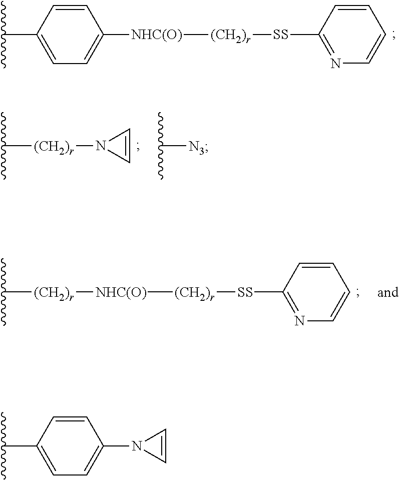

in which each r is independently selected from the integers from 1 to 10; G is a halogen; and R³⁰ and R³¹ are members independently selected from H and halogen and at least one of R³⁰ and R³¹ is halogen.

The reactive functional groups can be chosen such that they do not participate in, or interfere with, the reactions necessary to assemble or utilize the polypeptide conjugate. Alternatively, a reactive functional group can be protected from participating in the reaction by the presence of a protecting group. Those of skill in the art understand how to protect a particular functional group such that it does not interfere with a chosen set of reaction conditions. For examples of useful protecting groups, see, for example, Greene et al., PROTECTIVE GROUPS IN ORGANIC SYNTHESIS, John Wiley & Sons, New York, 1991.

### EXEMPLARY EMBODIMENTS

The various components of this reaction will be described in greater detail herein. Briefly, the fusion protein is a fusion of the target protein (i.e., the protein which is to be labeled) and an acceptor polypeptide (i.e., the peptide sequence that acts as a substrate for the lipoic acid ligase). If the method is performed in vivo, the nucleic acid sequence encoding the fusion protein may be introduced into the cell and transcription and translation allowed to occur. In some embodiments, the fusion protein may be present in a cell in a subject. In some embodiments, the fusion protein may be present in a transgenic subject. If the method is performed in vitro, the fusion protein may simply be added to the reaction mixture.

As used herein, polypeptide labeling in vitro means labeling of a polypeptide in a cell free environment. As an example, such a protein can be combined with a lipoic acid ligase and a lipoic acid analog under appropriate conditions and thereby labeled, in for example a test tube or a well of a multiwell plate.

As used herein, polypeptide labeling in vivo means labeling of a polypeptide in the context of a cell. The method can be used to label polypeptides that are intracellular polypeptides or cell surface polypeptides. The cell may be present in a subject (e.g., an insect such as Drosophila, a rodent such as a mouse, a human, and the like) or it may be present in culture. In some embodiments, a subject may be a transgenic subject.

A lipoic acid ligase or mutant thereof may also be expressed by the cell in some instances. In other instances, however, the lipoic acid ligase or mutant thereof may simply be added to the reaction mixture (if in vitro) or to the cell (if the target protein is a cell surface protein and the acceptor peptide is located on the extracellular domain of the target protein).

In an exemplary embodiment, the invention provides a Lp1A-mediated labeling of a polypeptide with an [¹⁸F]-prosthetic differing from a known substrate for this enzyme. The range of potential [¹⁸F]-prosthetic structures available imparts valuable synthetic flexibility to the methodology. The K_{M} of Lp1A is relatively low (13.3 μM ) (Puthenveetil, et al., J. Am. Chem. Soc. 2009, 131(45):16430), leading to high bioconjugation yields at low protein concentrations, generating [¹⁸F]-radiotracers with high specific activities.

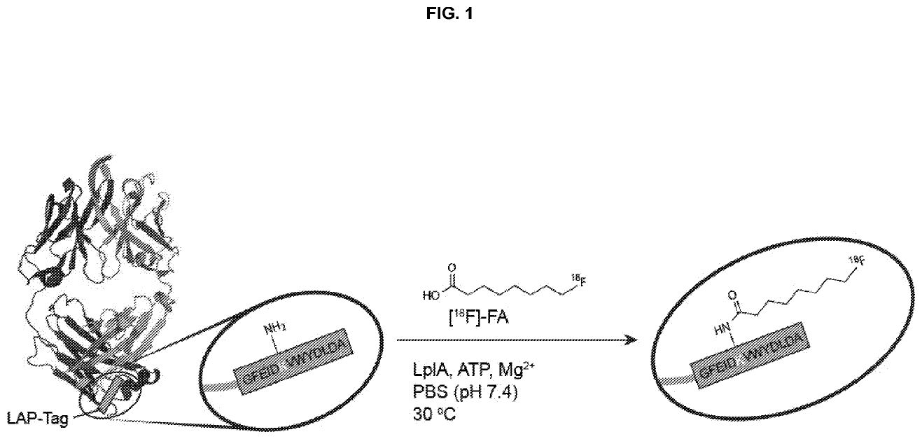

An exemplary strategy for realizing enzymatic radiofluorination of proteins is summarized in FIG. 1. A representative prosthetic is [¹⁸F]-8-fluorooctanoic acid ([¹⁸F]-FA) (Nagatsugi, et al., Nucl. Med. Biol. 1994, 21(6):809). 2G10, a recombinant human Fab antibody fragment is an exemplary tissue specific antibody having high affinity (K_{D}<50 mM) for the urokinase plasminogen activator receptor (uPAR)(Lebeau, et al., Cancer Res. 2013, 73(7):2070; Duriseti, et al., J. Biol. Chem. 2010, 285(35):26878; LeBeau, et al., Theranostics 2014, 4(3):267).

According to the method, the lipoic acid ligase or mutant thereof conjugates the labeled lipoic acid prosthetic to the acceptor polypeptide that is fused (either at the nucleic acid level or post-translationally) to the target protein. The method is independent of the protein type and thus any protein can be labeled in this manner. The product of this labeling reaction may or may not be directly detectable however depending upon the nature of the lipoic acid analog, as described herein. Accordingly, it may be necessary to react the conjugated lipoic acid analog with a detectable label. If the method is performed in vivo, the detectable label may be one capable of diffusion into a cell. If the method is used to label a cell surface protein, then the lipoic acid analog may be labeled with a membrane impermeant label in order to reduce entry and accumulation of the label intracellularly. The lipoic acid analog may be labeled prior to or after conjugation to the fusion protein.

Labeling of proteins allows one to track the movement and activity of such proteins. It also allows cells expressing such proteins to be tracked and imaged, as the case may be. The methods can be used in cells from virtually any organism including insect, yeast, frog, worm, fish, rodent, human and the like.

The method can be used to label virtually any protein. Examples include but are not limited to signal transduction proteins (e.g., cell surface receptors, kinases, adapter proteins), nuclear proteins (transcription factors, histones), mitochondrial proteins (cytochromes, transcription factors) and hormone receptors.

Lipoic acid ligase is an enzyme that catalyzes the ATP-dependent ligation of the small molecule lipoic acid to a specific lysine sidechain within one of three natural acceptor proteins E2p, E2o, and H-protein. As used herein, wild-type lipoic acid ligase refers to a naturally occurring lipoic acid ligase having wild-type lipoic acid ligase activity, or to a homolog thereof. An exemplary Lp1A is the GenBank sequence set forth as Accession No. AAA21740. An exemplary nucleotide sequence of E. coli wild-type lipoic acid ligase is GenBank Accession No. L27665.

Lipoic acid ligase is also known as lipoate-protein ligase A, Lp1A, and lipoate-protein ligase. In some embodiments of the invention, the lipoic acid ligase is an E. coli lipoic acid ligase, such as Lp1A. Homologs of E. coli lipoic acid ligase include, but are not limited to: Thermoplasma acidophilum Lp1A; Plasmodium falciparum LipL1, or LipL2; Oryza Sativa Lp1A (rice); Streptococcus pneumoniae Lp1A; and homologs from Pyrococcus horikoshii; Saccharomyces cerevisiae, Trypanosoma cruzi, Bacillus subtilis, and Leuconostoc mesenteroides. Homologs of E. coli lipoic acid ligase as well as mutants of such homologs are useful in methods and compositions of the invention.

The reaction between wild-type lipoic acid ligase and its substrate (discussed below) is referred to as orthogonal. This means that neither the ligase nor its substrate react with any other enzyme or molecule when present either in their native environment (i.e., a bacterial cell) or more importantly for the purposes of the invention in a non-native environment (e.g., a mammalian cell). Accordingly, the invention takes advantage of the high degree of specificity that has evolved between wild-type lipoic acid ligase and its substrate. Ligation interactions of the invention may or may not be orthogonal ligation reactions, it is not required that the ligation reactions of the invention be orthogonal. The only known natural substrates in bacteria of wild-type E. coli lipoic acid ligase are E2p, E2o, and H-protein, which are ligated to lipoic acid by the enzyme. The natural reaction of Lp1A has now been redirected such that unnatural structures, dissimilar to lipoic acid, can be ligated to either the natural protein substrates or Lp1A, or engineered peptide substrates.

A 12-17 amino acid minimal substrate sequence encompasses a lysine lipoylation site at the tip of a sharp β-turn in the substrate (e.g., such as E2o, E2p, or H-protein). For example in E. coli E2o, the lysine at the tip of a sharp β-turn is the lysine that is in position 44 of E. coli E2o, see GenBank Accession No. AAA23898. In each of the three lipoyl domains of E. coli E2p, the lysines at the tip of the sharp β-turn are the lysine lipoylation sites (e.g., the lysine in position of the lipoyl hybrid domain, see ProteinDataBank Accession No. 1QJO). In E. coli H-protein, the lysine at the tip of a sharp β-turn is the lysine that is in position 65 of E. coli H-protein, see GenBank Accession No. CAA52145. Testing has shown that although accurate positioning of the target lysine within the β-turn is important for Lp1A recognition, the residues flanking the lysine can be varied.

An exemplary acceptor peptide has an amino acid sequence of Xaa₁ Xaa₂ Xaa₃ Xaa₄ Xaa₅ Xaa₆ Xaa₇ Xaa₈ Xaa₉ Xaa₁₀ Xaa₁₁ Xaa₁₂ Xaa₁₃ . . . Xaaₓ, wherein the polypeptide Xaa₁, Xaaₓ is any combination of amino acids that results in the structure of the polypeptide suitable for use in methods and compositions of the invention. In an exemplary 13 amino acid acceptor polypeptide core sequence certain amino acids may be highly conserved among species.

In exemplary embodiments, the acceptor peptide comprises the amino acid sequence of a polypeptide having SEQ ID NO:2 (GFEIDKVWYDLDA), or a variant thereof. An exemplary variant polypeptide may include a portion of the amino acid sequence set forth herein as SEQ ID NO:2, (e.g., may be a 12, 13, or 14 amino acid portion as long as it includes the lysine residue and functions as an acceptor polypeptide), or may include the full sequence of one of SEQ ID NO:2 with additional amino acids attached at one or both ends of the polypeptide. As long as a polypeptide includes the positioning of the target lysine within the β-turn such that the polypeptide functions as a substrate for lipoic acid enzyme as described herein, (e.g., wild-type, homolog, and/or mutants thereof) the remainder of the polypeptide sequence can vary. A functional variant of an acceptor polypeptide may include an amino acid sequence that has up to 85%, 90%, 95%, or 99% identity to at least one of SEQ ID NO: 2.

One of ordinary skill in the art will recognize how to identify acceptor polypeptides and how to modify acceptor polypeptides of the invention to prepare additional acceptor polypeptides that are useful in methods and compositions of the invention. Various assays can be used to test the sequence specificity of acceptor polypeptides and their suitability for mammalian cell labeling applications. A non-limiting example of a method for identifying an acceptor polypeptide includes combining a candidate acceptor polypeptide with a labeled lipoic acid or analog thereof in the presence of a lipoic acid ligase or mutant thereof and determining a level of lipoic acid or lipoic acid analog incorporation, wherein lipoic acid or lipoic acid analog incorporation is indicative of a candidate acceptor polypeptide having specificity for a lipoic acid ligase or mutant thereof.

The acceptor peptide is used in the methods of the invention to tag target proteins that are to be labeled by lipoic acid ligase and mutants thereof. The acceptor peptide and target protein may be fused to each other either at the nucleic acid or amino acid level. Recombinant DNA technology for generating fusion nucleic acids that encode both the target protein and the acceptor peptide are known in the art. Additionally, the acceptor peptide may be fused to the target protein post-translationally. Such linkages may include cleavable linkers or bonds which can be cleaved once the desired labeling is achieved. Such bonds may be cleaved by exposure to a particular pH, or energy of a certain wavelength, and the like. Cleavable linkers are known in the art. Examples include thiol-cleavable cross-linker 3,3′-dithiobis(succinimidyl proprionate), amine-cleavable linkers, and succinyl-glycine spontaneously cleavable linkers.

The acceptor peptide can be fused to the target protein at any position. In some instances, it is preferred that the fusion not interfere with the activity of the target protein, accordingly, the acceptor peptide is fused to the protein at positions that do not interfere with the activity of the protein. Generally, the acceptor peptides can be C- or N-terminally fused to the target proteins. In still other instances, it is possible that the acceptor peptide is fused to the target protein at an internal position (e.g., a flexible internal loop). These proteins are then susceptible to specific tagging by lipoic acid ligase and/or mutants thereof in vivo and in vitro. This specificity is possible because neither lipoic acid ligase nor the acceptor peptide react with any other enzymes or peptides in a cell. One of ordinary skill in the art will understand how the amino acid sequence can be varied and how to vary the sequence such that it functions as an acceptor polypeptide for the methods and compositions of the invention. Acceptor peptides can be synthesized using standard peptide synthesis techniques. One of ordinary skill in the art will also recognize how to prepare an acceptor polypeptide such that is it attached (fused) to a target protein using routine methods.

Site-specific and target-oriented delivery of diagnostic and therapeutic agents is desirable for the purpose of detecting and treating a wide variety of human diseases, such as different types of malignancies and certain neurological disorders. Such procedures are accompanied by fewer side effects and a higher efficacy of drug. Various principles have been relied on in designing these delivery systems. For a review, see Garnett, Advanced Drug Delivery Reviews 53:171-216 (2001).

For tissue specific delivery, the discovery of tumor surface antigens has made it possible to develop delivery approaches where tumor cells displaying definable surface antigens are specifically targeted and detected and/or killed. There are three main classes of therapeutic monoclonal antibodies (MAb) that have demonstrated effectiveness in human clinical trials in treating malignancies: (1) unconjugated MAb, which either directly induces growth inhibition and/or apoptosis, or indirectly activates host defense mechanisms to mediate antitumor cytotoxicity; (2) drug-conjugated MAb, which preferentially delivers a potent cytotoxic toxin to the tumor cells and therefore minimizes the systemic cytotoxicity commonly associated with conventional chemotherapy; and (3) radioisotope-conjugated MAb, which delivers a sterilizing dose of radiation to the tumor. See review by Reff et al., Cancer Control 9:152-166 (2002). As will be appreciated by those of skill in the art, these motifs are equally applicable to MAbs bearing diagnostic agents conjugated through an acceptor polypeptide such as provided by the instant invention.

In order to arm MAbs with the power to detect and/or kill malignant cells, the MAbs can be connected to a detectable label or toxin, respectively, which is a component of the prosthetic. Exemplary toxins are obtained from a plant, bacterial, or fungal source, to form chimeric proteins called immunotoxins. Frequently used plant toxins are divided into two classes: (1) holotoxins (or class II ribosome inactivating proteins), such as ricin, abrin, mistletoe lectin, and modeccin, and (2) hemitoxins (class I ribosome inactivating proteins), such as pokeweed antiviral protein (PAP), saporin, Bryodin 1, bouganin, and gelonin. Commonly used bacterial toxins include diphtheria toxin (DT) and Pseudomonas exotoxin (PE). Kreitman, Current Pharmaceutical Biotechnology 2:313-325 (2001). Such toxins can be conjugated to the lipoid acid prosthetic and this assembly bound to the acceptor peptide of an acceptor-MAb fusion.

Conventional immunotoxins contain a MAb chemically conjugated to a toxin that is mutated or chemically modified to minimized binding to normal cells. Examples include anti-B4-blocked ricin, targeting CDS; and RFB4-deglycosylated ricin A chain, targeting CD22. Recombinant immunotoxins developed more recently are chimeric proteins consisting of the variable region of an antibody directed against a tumor antigen fused to a protein toxin using recombinant DNA technology. The toxin is also frequently genetically modified to remove normal tissue binding sites but retain its cytotoxicity. A large number of differentiation antigens, overexpressed receptors, or cancer-specific antigens have been identified as targets for immunotoxins, e.g., CD19, CD22, CD20, IL-2 receptor (CD25), CD33, IL-4 receptor, EGF receptor and its mutants, ErB2, Lewis carbohydrate, mesothelin, transferrin receptor, GM-CSF receptor, Ras, Bcr-Abl, and c-Kit, for the treatment of a variety of malignancies including hematopoietic cancers, glioma, and breast, colon, ovarian, bladder, and gastrointestinal cancers. See e.g., Brinkmann et al., Expert Opin. Biol. Ther. 1:693-702 (2001); Perentesis and Sievers, Hematology/Oncology Clinics of North America 15:677-701 (2001).

MAbs conjugated with radioisotope are used as another means of detecting and/or treating human malignancies with a high level of specificity and effectiveness. An exemplary detectable radioisotope is ¹⁸F. The most commonly used isotopes for therapy are the high-energy -emitters, such as ¹³¹I and ⁹⁰Y. Recently, ²¹³Bi-labeled anti-CD33 humanized MAb has also been tested in phase I human clinical trials. Reff et al., supra.

A number of MAbs have been used for therapeutic purposes. For example, the use of rituximab (Rituxan™), a recombinant chimeric anti-CD20 MAb, for treating certain hematopoietic malignancies was approved by the FDA in 1997. Other MAbs that have since been approved for therapeutic uses in treating human cancers include: alemtuzumab (Campath-1H™), a humanized rat antibody against CD52; and gemtuzumab ozogamicin (Mylotarg™), a calicheamicin-conjugated humanized mouse antCD33 MAb. The FDA is also currently examining the safety and efficacy of several other MAbs for the purpose of site-specific delivery of cytotoxic agents or radiation, e.g., radiolabeled Zevalin™ and Bexxar™.

In various embodiments, the invention provides a method of conjugating a labeled prosthetic to a MAb using Lp1A, and conjugates prepared according to this method.

A second important consideration in designing a diagnostic or drug delivery system is the accessibility of a target tissue to a therapeutic agent. This is an issue of particular concern in the case of detecting and/or treating a disease of the central nervous system (CNS), where the blood-brain barrier prevents the diffusion of macromolecules. Several approaches have been developed to bypass the blood-brain barrier for effective delivery of therapeutic agents to the CNS.

The understanding of iron transport mechanism from plasma to brain provides a useful tool in bypassing the blood-brain barrier (BBB). Iron, transported in plasma by transferrin, is an essential component of virtually all types of cells. The brain needs iron for metabolic processes and receives iron through transferrin receptors located on brain capillary endothelial cells via receptor-mediated transcytosis and endocytosis. Moos and Morgan, Cellular and Molecular Neurobiology 20:77-95 (2000). Delivery systems based on transferrin-transferrin receptor interaction have been established for the efficient delivery of peptides, proteins, and liposomes into the brain. For example, peptides can be coupled with a Mab directed against the transferrin receptor to achieve greater uptake by the brain, Moos and Morgan, Supra. Similarly, when coupled with an MAb directed against the transferrin receptor, the transportation of basic fibroblast growth factor (bFGF) across the blood-brain barrier is enhanced. Song et al., The Journal of Pharmacology and Experimental Therapeutics 301:605-610 (2002); Wu et al., Journal of Drug Targeting 10:239-245 (2002). In addition, a liposomal delivery system for effective transport of the chemotherapy drug, doxorubicin, into C6 glioma has been reported, where transferrin was attached to the distal ends of liposomal PEG chains. Eavarone et al., J. Biomed. Mater. Res. 51:10-14 (2000). A number of US patents also relate to delivery methods bypassing the blood-brain barrier based on transferrin-transferrin receptor interaction. See e.g., U.S. Pat. Nos. 5,154,924; 5,182,107; 5,527,527; 5,833,988; 6,015,555.

There are other suitable conjugation partners for a pharmaceutical agent to bypass the blood-brain barrier. For example, U.S. Pat. Nos. 5,672,683, 5,977,307 and WO 95/02421 relate to a method of delivering a neuropharmaceutical agent across the blood-brain barrier, where the agent is administered in the form of a fusion protein with a ligand that is reactive with a brain capillary endothelial cell receptor; WO 99/00150 describes a drug delivery system in which the transportation of a drug across the blood-brain barrier is facilitated by conjugation with an MAb directed against human insulin receptor; WO 89/10134 describes a chimeric peptide, which includes a peptide capable of crossing the blood brain barrier at a relatively high rate and a hydrophilic neuropeptide incapable of transcytosis, as a means of introducing hydrophilic neuropeptides into the brain; WO 01/60411 A1 provides a pharmaceutical composition that can easily transport a pharmaceutically active ingredient into the brain. The active ingredient is bound to a hibernation-specific protein that is used as a conjugate, and administered with a thyroid hormone or a substance promoting thyroid hormone production. In addition, an alternative route of drug delivery for bypassing the blood-brain barrier has been explored. For instance, intranasal delivery of therapeutic agents without the need for conjugation has been shown to be a promising alternative delivery method (Frey, 2002, Drug Delivery Technology, 2(5):46-49).

In addition to facilitating the transportation of drugs across the blood-brain barrier, transferrin-transferrin receptor interaction is also useful for specific targeting of certain tumor cells, as many tumor cells overexpress transferrin receptor on their surface. This strategy has been used for delivering bioactive macromolecules into K562 cells via a transferrin conjugate (Wellhoner et al., The Journal of Biological Chemistry 266:4309-4314 (1991)), and for delivering insulin into enterocyte-like Caco-2 cells via a transferrin conjugate (Shah and Shen, Journal of Pharmaceutical Sciences 85:1306-1311 (1996)).

Furthermore, as more becomes known about the functions of various iron transport proteins, such as lactotransferrin receptor, melanotransferrin, ceruloplasmin, and Divalent Cation Transporter and their expression pattern, some of the proteins involved in iron transport mechanism (e.g., melanotransferrin), or their fragments, have been found to be similarly effective in assisting therapeutic agents transport across the blood-brain barrier or targeting specific tissues (WO 02/13843 A2, WO 02/13873 A2). For a review on the use of transferrin and related proteins involved in iron uptake as conjugates in drug delivery, see Li and Qian, Medical Research Reviews 22:225-250 (2002).

The concept of tissue-specific delivery of therapeutic agents goes beyond the interaction between transferrin and transferrin receptor or their related proteins. For example, a bone-specific delivery system has been described in which proteins are conjugated with a bone-seeking aminobisphosphate for improved delivery of proteins to mineralized tissue. Uludag and Yang, Biotechnol. Prog. 18:604-611 (2002). For a review on this topic, see Vyas et al., Critical Reviews in Therapeutic Drug Carrier System 18:1-76 (2001).

A variety of linkers may be used in the process of generating bioconjugates for the purpose of specific delivery of diagnostic and/or therapeutic agents. Suitable linkers include homo- and heterobifunctional cross-linking reagents, which may be cleavable by, e.g., acid-catalyzed dissociation, or non-cleavable (see, e.g., Srinivasachar and Neville, Biochemistry 28:2501-2509 (1989); Wellhoner et al., The Journal of Biological Chemistry 266:4309-4314 (1991)). Interaction between many known binding partners, such as biotin and avidin/streptavidin, can also be used as a means to join a therapeutic agent and a conjugate partner that ensures the specific and effective delivery of the therapeutic agent. Using the methods of the invention, proteins may be used to deliver molecules to intracellular compartments as conjugates. Proteins, peptides, hormones, cytokines, small molecules or the like that bind to specific cell surface receptors that are internalized after ligand binding may be used for intracellular targeting of conjugated therapeutic compounds. Typically, the receptor-ligand complex is internalized into intracellular vesicles that are delivered to specific cell compartments, including, but not limited to, the nucleus, mitochondria, golgi, ER, lysosome, and endosome, depending on the intracellular location targeted by the receptor. By conjugating the receptor ligand with the desired molecule, the drug will be carried with the receptor-ligand complex and be delivered to the intracellular compartments normally targeted by the receptor. The drug can therefore be delivered to a specific intracellular location in the cell where it is needed to treat a disease.

Many proteins may be used to target therapeutic agents to specific tissues and organs. Targeting proteins include, but are not limited to, growth factors (EPO, HGH, EGF, nerve growth factor, FGF, among others), cytokines (GM-CSF, G-CSF, the interferon family, interleukins, among others), hormones (FSH, LH, the steroid families, estrogen, corticosteroids, insulin, among others), serum proteins (albumin, lipoproteins, fetoprotein, human serum proteins, antibodies and fragments of antibodies, among others), and vitamins (folate, vitamin C, vitamin A, among others). Targeting agents are available that are specific for receptors on most cells types.

In various embodiments, the invention provides a method of conjugating a labeled prosthetic to a MAb using Lp1A, and conjugates prepared according to this method. These conjugates are capable of targeting specific tissues and being delivered to these tissues.

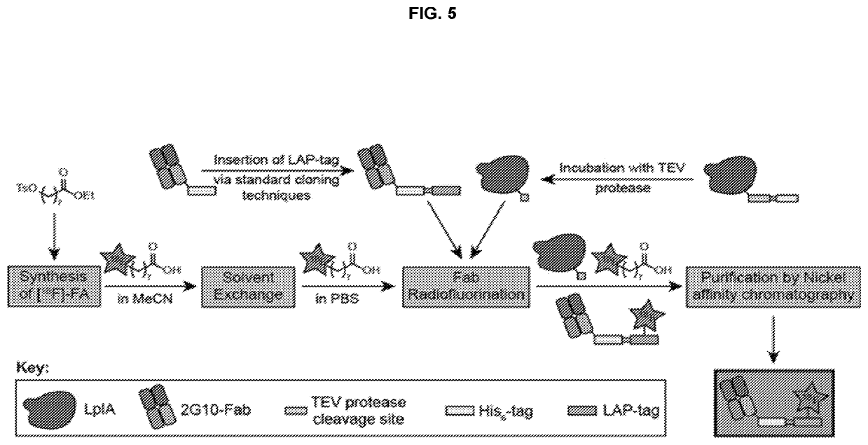

The invention is also directed in part to the identification and use of analogs of lipoic acid in assays and methods of the invention such as those described herein. As described herein, Lp1A naturally catalyzes the ATP-dependent ligation of the small-molecule lipoic acid to a specific lysine sidechain within one of three natural acceptor proteins (E2p, E2o, and. H-protein). As depicted in FIG. 5, Lp1A has been redirected to ligate analogs of lipoic acid, in order to label proteins with useful biophysical probes. A number of alkyl azide and alkyne Lp1A substrates of varying lengths have been synthesized.

One of ordinary skill in the art will recognize how to modify lipoic acid prosthetics of the invention to prepare additional lipoic acid analogs that are useful in methods and compositions of the invention. Various assays can be used to test the sequence specificity of Lp1A, and the suitability of various lipoic acid analogs and acceptor polypeptides for mammalian cell labeling applications. A non-limiting example of a method for identifying a lipoic acid analog having specificity for a lipoic acid ligase or a mutant includes combining an acceptor polypeptide with a candidate lipoic acid analog molecule in the presence of a lipoic acid ligase or mutant thereof and determining the presence of lipoic acid analog incorporation, wherein lipoic acid analog incorporation is indicative of a candidate lipoic acid analog having specificity for a lipoic acid ligase or mutant thereof. Additional exemplary assays and methods of determining the presence of lipoic acid incorporation are provided in the Examples section herein.

In some aspects of the invention, an azide group that has been attached to the target can be selectively derivatized to any fluorescent probe conjugated to a cyclooctyne reaction partner. The azide group is thus useful as a “functional group handle.” Direct ligation of a fluorophore may be used as a labeling procedure, but incorporation of a “functional group handle” is more feasible due to the small size of the lipoate binding pocket, and provides greater versatility for subsequent incorporation of probes of any structure. Many functional group handles have been used in chemical biology, including ketones, organic azides, and alkynes (Prescher, J. A. & Bertozzi, C. R. 2005 Nat. Chem. Biol. 1, 13-21). Organic azides are suitable for live cell applications, because the azide group is both abiotic and non-toxic in animals and can be selectively derivatized under physiological conditions (without any added metals or cofactors) with cyclooctynes, which are also unnatural (Agard, N. J., et. al., 2006 ACS Chem. Biol. 1, 644-648). Methods of using functional group handles such as azides and alkynes are well known in the art and methods and procedures for the use of such functional group handles in combination with a cyclooctyne reaction a partner are understood and can be practiced by those of ordinary skill in the art using routine techniques.

Thus, in various embodiments, the invention provides labeled lipoic acid prosthetics, which are substrates for Lp1A and are capable of being conjugated to an acceptor peptide using Lp1A.

The invention is directed in part to generating lipoic acid ligase mutants that recognize lipoic acid analogs and conjugate such analogs to the acceptor peptide. Lipoic acid ligase mutants can be generated in any number of ways, including in vitro compartmentalization, genetic selections, yeast display, or FACS in mammalian cells, described in greater detail herein, all of which are standard methods understood and routinely practiced by those of ordinary skill in the art.

Labeling methods of the invention rely on the activity of lipoic acid ligase and mutants thereof that recognize and conjugate lipoic acid analogs onto fusion proteins via the . acceptor peptide. The invention provides lipoic acid ligase mutants that recognize lipoic acid analogs. As used herein, a lipoic acid ligase mutant is a variant of lipoic acid ligase that is enzymatically active towards a lipoic acid analog (such as those described herein). As used herein, “enzymatically active” means that the mutant is able to recognize and conjugate a lipoic acid analog to the acceptor peptide.

A lipoic acid ligase mutant of use in the invention can have various mutations, including addition, deletion or substitution of one or more amino acids. Preferably, the mutation will be present in the lipoic acid interaction and activation region, spanning amino acids 16-149. Generally, these mutants will possess one or more amino acid substitutions relative to the wild-type lipoic acid ligase amino acid sequence (SEQ ID NO:1). In most instances, the lipoic acid ligase mutants do not comprise an amino acid substitution (or other form of mutation) of the lysine that corresponds to lysine 133 of the wild-type E. coli lipoic acid ligase set forth as SEQ ID NO:1 (which is the putative catalytic residue).

A lipoic acid ligase mutant may retain some level of activity for lipoic acid or an analog thereof. Its binding affinity for lipoic acid or an analog thereof may be similar to that of wild-type lipoic acid ligase. Preferably, the mutant has higher binding affinity for a lipoic acid analog than it does for lipoic acid. Consequently, lipoic acid conjugation to an acceptor peptide would be lower in the presence of a lipoic acid analog. In still other embodiments, the lipoic acid ligase mutant has no binding affinity for lipoic acid.

In some embodiments of the invention, a lipoic acid ligase analog may have a nucleic acid sequence that has up to 85%, 90%, 95%, or 99% identity to the nucleic acid sequence of a wild-type lipoic acid ligase and ligates lipoic acid and/or a lipoic acid prosthetic to an acceptor polypeptide. A lipoic acid ligase analog (mutant) may include an amino acid sequence that has up 85%, 90%, 95%, or 99% identity to the amino acid sequence of wild-type E. coli lipoic acid ligase (e.g., to SEQ ID NO:1) and will retain function as a lipoic acid ligase in methods of the invention. In some embodiments, a lipoic acid ligase used in methods of the invention is the lipoic acid ligase having the sequence set forth as SEQ ID NO:1.

One of ordinary skill in the art will recognize how to identify suitable lipoic acid ligases and how to modify lipoic acid ligases of the invention to prepare additional lipoic acid ligases that are useful in methods and compositions of the invention. Various assays can be used to test the specificity and functionality of a lipoic acid ligase and its suitability for mammalian cell labeling applications. A non-limiting example of a method for identifying a lipoic acid ligase includes contacting a lipoic acid or lipoic acid analog with an acceptor polypeptide in the presence of a candidate lipoic acid ligase molecule, and detecting a lipoic acid or lipoic acid analog that is bound to the acceptor polypeptide, wherein the presence of a lipoic acid or lipoic acid analog bound to an acceptor polypeptide indicates that the candidate lipoic acid ligase molecule is a lipoic acid ligase that has specificity for the lipoic acid or lipoic acid analog.

Lipoic acid incorporation can be measured using .sup.3H-lipoic acid and measuring incorporation of radioisotope in the peptide. Conjugation of the lipoic acid analog to an acceptor peptide can be assayed by various methods including, but not limited to, HPLC or mass-spec assays, as described herein and as shown in the figures herein.

The skilled artisan will realize that conservative amino acid substitutions may be made in lipoic acid ligase mutants to provide functionally equivalent variants, i.e., the variants retain the functional capabilities of the particular lipoic acid ligase mutant. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

Conservative amino-acid substitutions in the amino acid sequence of lipoic acid ligase mutants to produce functionally equivalent variants typically are made by alteration of a nucleic acid encoding the mutant. Such substitutions can be made by a variety of methods known to one of ordinary skill in the art. For example, amino acid substitutions may be made by PCR-directed mutation, site-directed mutagenesis according to the method of Kunkel (Kunkel, PNAS 82: 488-492, 1985), or by chemical synthesis of a nucleic acid molecule encoding a lipoic acid ligase mutant.

Similarly, lipoic acid ligase mutants can be made using standard molecular biology techniques known to those of ordinary skill in the art. For example, the mutants may be formed by transcription and translation from a nucleic acid sequence encoding the mutant. Such nucleic acid sequences can be made based on the teaching of wild-type lipoic acid ligase sequence and the position and type of amino acid substitution.

Thus, in various embodiments, the invention provides methods of producing lipoic acid ligase mutants, characterizing them and using them to prepare conjugates between labeled lipoic acid prosthetics and an acceptor protein, or an acceptor protein region of a fusion protein further comprising a tissue selective targeting region.

The invention further provides methods for screening candidate molecules for activity as a lipoic acid ligase mutant. These screening methods can also be combined with methods for generating candidates. Exemplary methods include, but are not limited to, in vitro compartmentalization, life/death selections in bacteria, yeast display, or FACS in mammalian cells, each of which is known and routinely used by those of ordinary skill in the art. In vitro compartmentalization (IVC) selection strategy provides a platform to conduct multiple turnover selection for enzymes. In this completely in vitro system genes are compartmentalized by forming a water-in-oil emulsion. In this water-in-oil emulsion compartment genotype-phenotype linkage is maintained through out the entire process from transcription/translation to substrate to product formation. The main advantage of IVC over other traditional methods of selection is its ability to select out faster enzymes from slower enzymes.

In various embodiments, the presence on the fusion protein of the detectable label of the lipoic acid prosthetic after incubation of the fusion protein with the mutated Lp1A and the lipoid acid prosthetic is indicative or confirmatory of the activity of the mutant Lp1A.

The following is an example of a genetic selection strategy that may be used to evolve lipoic acid ligase mutants. In the method, the selection is based on an E. coli strain with knock out Lp1A and LipB gene. This allows the strain to grow only in presence of succinate plus acetate or by introducing a functional Lp1A mutant that recognizes an exogenous lipoic acid prosthetic as its substrate. For selection an Lp1A mutant library may be transformed to this strain and will allow it to grow in presence of a suitable molar ratio of the lipoic acid prosthetic. Mutants that recognize the prosthetic will grow but mutants that do not recognize it will cease to grow. β-lactam-based antibiotic will selectively kill the dividing bacteria (carrying mutants that are not of interest). The remaining static pool of bacteria (carrying Lp1A mutants that are of interest) are harvested and used for successive round of selections.

The labeling methods of the invention further rely on lipoic acid prosthetics that are recognized and conjugated to acceptor peptides by lipoic acid ligase mutants.

The lipoic acid ligase mutants are preferably capable of recognizing and conjugating lipoic acid analogs to acceptor peptides, in a manner similar to that in which wild-type lipoic acid ligase recognizes and conjugates lipoic acid to the acceptor peptide.

In various embodiments, the lipoic acid prosthetic binds to a lipoic acid ligase mutant and it preferably binds with an affinity at least that of or comparable to the binding affinity of wild-type lipoic acid ligase to lipoic acid. However, lipoic acid prosthetics that bind with lower affinities are still useful according to the invention. In some embodiments, the lipoic acid prosthetic is not recognized by wild-type lipoic acid ligase derived from either E. coli or from other cell types (e.g., the cell in which the labeling reaction is proceeding) but is recognized by the mutant.

Some lipoic acid prosthetics are not themselves directly detectable, while others are. In the case of the former type, the lipoic acid prosthetic undergoes reaction with another moiety (after conjugation to the acceptor peptide), thereby becoming detectable. The subsequent modification of this former type of lipoic acid analog is referred to as a bio-orthogonal ligation reaction and it is used to couple (i.e., label) these lipoic acid prosthetics to detectable labels such as radioisotopes and fluorophores. An exemplary chemistry type for performing orthogonal liggation is Click chemistry. The alkyne/azide [3+2] cycloaddition chemistry, based on Click chemistry (Wang et al. J. Am. Chem. Soc. 125:11164-11165, 2003), is also specific, in part because the two reactive partners do not have cellular counterparts (i.e., the two functional groups are non-naturally occurring). Nonlimiting examples of fluorophores that may be conjugated to a cyclooctyne are Alexa Fluor 568 and Cy3.

As stated above, other lipoic acid prosthetics may be themselves directly detectable, e.g., comprise a detectable label, e.g. a radioisotope, e.g., ¹⁸F or a fluorophore. Examples of lipoic acid prosthetics conjugated to fluorophores include but are not limited to those conjugated to coumarin, fluorescein, aryl azides, diazirines, benzophenones, resorufins, various xanthene-type fluorophores, chloroalkanes, metal-binding ligands, or derivatives thereof.

A lipoic acid prosthetic can also be fluorogenic. As used herein, a fluorogenic compound is one that is not detectable (e.g., fluorescent) by itself, but when conjugated to another moiety becomes fluorescent. An example of this is non-fluorescent coumarin phosphine which reacts with azides to produce fluorescent coumarin. Fluorogenic lipoic acid analogs are especially useful to keeping background to a minimum (e.g., cellular imaging applications).

As stated above, the lipoic acid analogs can be conjugated to detectable labels, e.g., through conjugation using a Click chemistry reaction partner.

The labels can also be antibodies or antibody fragments or their corresponding antigen, epitope or hapten binding partners. Detection of such bound antibodies and proteins or peptides is accomplished by techniques well known to those skilled in the art. Antibody/antigen complexes which form in response to hapten conjugates are easily detected by linking a label to the hapten or to antibodies which recognize the hapten and then observing the site of the label. Alternatively, the antibodies can be visualized using secondary antibodies or fragments thereof that are specific for the primary antibody used. Polyclonal and monoclonal antibodies may be used. Antibody fragments include Fab, F(ab)₂, Fd and antibody fragments which include a CDR3 region. The conjugates can also be labeled using dual specificity antibodies.

The label can be a positron emission tomography (PET) isotope such as fluorine-18, carbon-11, iodine-124, zirconium-89, or gallium-68. The label can be a single photon emission computed tomography (SPECT) isotope such as iodine-123, technetium-99m or indium-111.

The label can also be an singlet oxygen radical generator including but not limited to resorufin, malachite green, fluorescein, benzidine and its analogs including 2-aminobiphenyl, 4-aminobiphenyl, 3,3′-diaminobenzidine, 3,3′-dichlorobenzidine, 3,3′-dimethoxybenzidine, and 3,3′-dimethylbenzidine. These molecules are useful in EM staining and can also be used to induce localized toxicity.

The label can also be an analyte-binding group such as but not limited to a metal chelator (e.g., a copper chelator). Examples of metal chelators include EDTA, EGTA, and molecules having pyridinium substituents, imidazole substituents, and/or thiol substituents. These labels can be used to analyze local environment of the target protein (e.g., Ca²⁺ concentration).

The label can also be a heavy atom carrier. Such labels would be particularly useful for X-ray crystallographic study of the target protein. Heavy atoms used in X-ray crystallography include but are not limited to Au, Pt and Hg. An example of a heavy atom carrier is iodine.

The label may also be a photoactivatable cross-linker. A photoactivable cross linker is a cross linker that becomes reactive following exposure to radiation (e.g., a ultraviolet radiation, visible light, etc.). Examples include benzophenones, aziridines, a photoprobe analog of geranylgeranyl diphosphate (2-diazo-3,3,3-trifluoropropionyloxy-farnesyl diphosphate or DATFP-FPP) (Quellhorst et al. J Biol. Chem. 2001 Nov. 2; 276(44):40727-33), a DNA analogue 5-[N-(p-azidobenzoyl)-3-aminoallyl]-dUTP (N(3)RdUTP), sulfosuccinimidyl-2(7-azido-4-methylcoumarin-3-acetamido)-ethyl-1,3′-dith-iopropionate (SAED) and 1-[N-(2-hydroxy-5-azidobenzoyl)-2-aminoethyl]-4-(N-hydroxysuccinimidyl)-s-uccinate.

The label may also be a photoswitch label. A photoswitch label is a molecule that undergoes a conformational change in response to radiation. For example, the molecule may change its conformation from cis to trans and back again in response to radiation. The wavelength required to induce the conformational switch will depend upon the particular photoswitch label. Examples of photoswitch labels include azobenzene, 3-nitro-2-naphthalenemethanol. Examples of photoswitches are also described in van Delden et al. Chemistry. 2004 Jan. 5; 10(1):61-70; van Delden et al. Chemistry. 2003 Jun. 16; 9(12):2845-53; Zhang et al. Bioconjug Chem. 2003 July-August;14(4):824-9; Irie et al. Nature. 2002 Dec. 19-26; 420(6917):759-60; as well as many others.

The label may also be a photolabile protecting group. Examples of photolabile protecting group include a nitrobenzyl group, a dimethoxy nitrobenzyl group, nitroveratryloxycarbonyl (NVOC), 2-(dimethylamino)-5-nitrophenyl (DANP), Bis(o-nitrophenyl)ethanediol, brominated hydroxyquinoline, and coumarin-4-ylmethyl derivative. Photolabile protecting groups are useful for photocaging reactive functional groups.

The label may comprise non-naturally occurring amino acids. Examples of non-naturally occurring amino acids include for glutamine (Glu) or glutamic acid residues: .alpha.-aminoadipate molecules; for tyrosine (Tyr) residues: phenylalanine (Phe), 4-carboxymethyl-Phe, pentafluoro phenylalanine (PfPhe), 4-carboxymethyl-L-phenylalanine (cmPhe), 4-carboxydifluoromethyl-L-phenylalanine (F.sub.2 cmPhe), 4-phosphonomethyl-phenylalanine (Pmp), (difluorophosphonomethyl)phenylalanine (F.sub.2Pmp), O-malonyl-L-tyrosine (malTyr or OMT), and fluoro-O-malonyltyrosine (FOMT); for proline residues: 2-azetidinecarboxylic acid or pipecolic acid (which have 6-membered, and 4-membered ring structures respectively); 1-aminocyclohexylcarboxylic acid (Ac.sub.6c); 3-(2-hydroxynaphtalen-1-yl)-propyl; S-ethylisothiourea; 2-NH.sub.2-thiazoline; 2-NH.sub.2-thiazole; asparagine residues substituted with 3-indolyl-propyl at the C terminal carboxyl group. Modifications of cysteines, histidines, lysines, arginines, tyrosines, glutamines, asparagines, prolines, and carboxyl groups are known in the art and are described in U.S. Pat. No. 6,037,134. These types of labels can be used to study enzyme structure and function.

The label may be an enzyme or an enzyme substrate. Examples of these include (enzyme (substrate)): Alkaline Phosphatase (4-Methylumbelliferyl phosphate Disodium salt; 3-Phenylumbelliferyl phosphate Hemipyridine salt); Aminopeptidase (L-Alanine-4-methyl-7-coumarinylamide trifluoroacetate; Z-L-arginine-4-methyl-7-coumarinylamide hydrochloride; Z-glycyl-L-proline-4-methyl-7-coumarinylamide); Aminopeptidase B (L-Leucine-4-methyl-7-coumarinylamide hydrochloride); Aminopeptidase M (L-Phenylalanine 4-methyl-7-coumarinylamide trifluoroacetate); Butyrate esterase (4-Methylumbelliferyl butyrate); Cellulase (2-Chloro-4-nitrophenyl-beta-D-cellobioside); Cholinesterase (7-Acetoxy-1-methylquinolinium iodide; Resorufin butyrate); alpha-Chymotrypsin, (Glutaryl-L-phenylalanine 4-methyl-7-coumarinylamide); N—(N-Glutaryl-L-phenylalanyl)-2-aminoacridone; N—(N-Succinyl-L-phenylalanyl)-2-aminoacridone); Cytochrome P450 2B6 (7-Ethoxycoumarin); Cytosolic Aldehyde Dehydrogenase (Esterase Activity) (Resorufin acetate); Dealkylase (O.sup.7-Pentylresorufin); Dopamine beta-hydroxylase (Tyramine); Esterase (8-Acetoxypyrene-1,3,6-trisulfonic acid Trisodium salt; 3-(2 Benzoxazolyl)umbelliferyl acetate; 8-Butyryloxypyrene-1,3,6-trisulfonicacid Trisodium salt; 2′,7′-Dichlorofluorescin diacetate; Fluorescein dibutyrate; Fluorescein dilaurate; 4-Methylumbelliferyl acetate; 4-Methylumbelliferyl butyrate; 8-Octanoyloxypyrene-1,3,6-trisulfonic acid Trisodium salt; 8-Oleoyloxypyrene-1,3,6-trisulfonic acid Trisodium salt; Resorufin acetate); Factor X Activated (Xa) (4-Methylumbelliferyl 4-guanidinobenzoate hydrochloride Monohydrate); Fucosidase, alpha-L-(4-Methylumbelliferyl-alpha-L-fucopyranoside); Galactosidase, alpha-(4-Methylumbelliferyl-alpha-D galactopyranoside); Galactosidase, beta-(6,8-Difluoro-4-methylumbelliferyl-beta-D-galactopyranoside; Fluorescein di(beta-D-galactopyranoside); 4-Methylumbelliferyl-alpha-D-galactopyranoside; 4-Methylumbelliferyl-beta-D-lactoside: Resorufin-beta-D-galactopyranoside; 4-(Trifluoromethyl)umbelliferyl-beta-D-galactopyranoside; 2-Chloro-4-nitrophenyl-beta-D-lactoside); Glucosaminidase, N-acetyl-beta-(4-Methylumbelliferyl-N-acetyl-beta-D-glucosaminide Dihydrate); Glucosidase, alpha-(4-Methylumbelliferyl-alpha-D-glucopyranoside); Glucosidase, beta-(2-Chloro-4-nitrophenyl-beta-D-glucopyranoside; 6,8-Difluoro-4-methylumbelliferyl-beta-D-glucopyranoside; 4-Methylumbelliferyl-beta-D-glucopyranoside; Resorufin-beta-D-glucopyranoside; 4-(Trifluoromethyl)umbelliferyl-beta-D-glucopyranoside); Glucuronidase, beta-(6,8-Difluoro-4-methylumbelliferyl-beta-D-glucuronide Lithium salt; 4-Methylumbelliferyl-beta-D-glucuronide Trihydrate); Leucine aminopeptidase (L-Leucine-4-methyl-7-coumarinylamide hydrochloride); Lipase (Fluorescein dibutyrate; Fluorescein dilaurate; 4-Methylumbelliferyl butyrate; 4-Methylumbelliferyl enanthate; 4-Methylumbelliferyl oleate; 4-Methylumbelliferyl palmitate; Resorufin butyrate); Lysozyme (4-Methylumbelliferyl-N,N′,N″-triacetyl-beta-chitotrioside); Mannosidase, alpha-(4-Methylumbelliferyl-alpha-D-mannopyranoside); Monoamine oxidase (Tyramine); Monooxygenase (7-Ethoxycoumarin); Neuraminidase (4-Methylumbelliferyl-N-acetyl-alpha-D-neuraminic acid Sodium salt Dihydrate); Papain (Z-L-arginine-4-methyl-7-coumarinylamide hydrochloride); Peroxidase (Dihydrorhodamine 123); Phosphodiesterase (1-Naphthyl4-phenylazophenyl phosphate; 2-Naphthyl4-phenylazophenyl phosphate); Prolyl endopeptidase (Z-glycyl-L-proline-4-methyl-7-coumarinylamide; Z-glycyl-L-proline-2-naphthylamide; Z-glycyl-L-proline-4-nitroanilide); Sulfatase (4-Methylumbelliferyl sulfate Potassium salt); Thrombin (4-Methylumbelliferyl 4-guanidinobenzoate hydrochloride Monohydrate); Trypsin (Z-L-arginine-4-methyl-7-coumarinylamide hydrochloride; 4-Methylumbelliferyl 4-guanidinobenzoate hydrochloride Monohydrate); Tyramine dehydrogenase (Tyramine).

The labels can be attached to the lipoic acid analogs either before or after the analog has been conjugated to the acceptor peptide, presuming that the label does not interfere with the activity of lipoic acid ligase. Labels can be attached to the lipoic acid analogs by any mechanism known in the art. Some of these mechanisms are already described above for particular analogs. Other examples of functional groups which are reactive with various labels include, but are not limited to, (functional group: reactive group of light emissive compound) activated ester:amines or anilines; acyl azide:amines or anilines; acyl halide:amines, anilines, alcohols or phenols; acyl nitrile:alcohols or phenols; aldehyde:amines or anilines; alkyl halide:amines, anilines, alcohols, phenols or thiols; alkyl sulfonate:thiols, alcohols or phenols; anhydride:alcohols, phenols, amines or anilines; aryl halide:thiols; aziridine:thiols or thioethers; carboxylic acid:amines, anilines, alcohols or alkyl halides; diazoalkane:carboxylic acids; epoxide:thiols; haloacetamide:thiols; halotriazine:amines, anilines or phenols; hydrazine:aldehydes or ketones; hydroxyamine:aldehydes or ketones; imido ester:amines or anilines; isocyanate:amines or anilines; and isothiocyanate:amines or anilines.

The labels are detected using a detection system. The nature of such detection systems will depend upon the nature of the detectable label. The detection system can be selected from any number of detection systems known in the art. These include a positron emission tomographic (PET) system, fluorescent detection system, a photographic film detection system, a chemiluminescent detection system, an enzyme detection system, an atomic force microscopy (AFM) detection system, a scanning tunneling microscopy (STM) detection system, an optical detection system, a nuclear magnetic resonance (NMR) detection system, a near field detection system, and a total internal reflection (TIR) detection system.

The invention provides in some instances lipoic acid ligase or mutant thereof and/or lipoic acid analogs in an isolated form. As used herein, an isolated lipoic acid ligase or mutant thereof is a lipoic acid ligase or mutant thereof that is separated from its native environment in sufficiently pure form so that it can be manipulated or used for any one of the purposes of the invention. Thus, isolated means sufficiently pure to be used (i) to raise and/or isolate antibodies, (ii) as a reagent in an assay, or (iii) for sequencing, etc.

Isolated lipoic acid prosthetics similarly are analogs that have been substantially separated from either their native environment (if it exists in nature) or their synthesis environment. Accordingly, the lipoic acid prosthetics are substantially separated from any or all reagents present in their synthesis reaction that would be toxic or otherwise detrimental to the target protein, the acceptor peptide, the lipoic acid ligase mutant, or the labeling reaction. Isolated lipoic acid analogs, for example, include compositions that comprise less than 25% contamination, less than 20% contamination, less than 15% contamination, less than 10% contamination, less than 5% contamination, or less than 1% contamination (w/w).

The invention further provides nucleic acids coding for lipoic acid ligase mutants and host cells expressing such nucleic acids. The nucleotide sequence of wild-type lipoic acid ligase is provided as SEQ ID NO: 1.One of ordinary skill in the art will be able to determine the codons corresponding to each of the amino acid residues recited herein.

The invention also embraces degenerate nucleic acids that differ from the mutant nucleic acid sequences provided herein in codon sequence due to degeneracy of the genetic code. For example, serine residues are encoded by the codons TCA, AGT, TCC, TCG, TCT and AGC. Each of the six codons is equivalent for the purposes of encoding a serine residue. Thus, it will be apparent to one of ordinary skill in the art that any of the serine-encoding nucleotide triplets may be employed to direct the protein synthesis apparatus, in vitro or in vivo, to incorporate a serine residue into an elongating mutant. Similarly, nucleotide sequence triplets which encode other amino acid residues include, but are not limited to: CCA, CCC, CCG and CCT (proline codons); CGA, CGC, CGG, CGT, AGA and AGG (arginine codons); ACA, ACC, ACG and ACT (threonine codons); AAC and AAT (asparagine codons); and ATA, ATC and ATT (isoleucine codons). Other amino acid residues may be encoded similarly by multiple nucleotide sequences.

The invention also involves expression vectors coding for lipoic acid ligase mutants and host cells containing those expression vectors. Virtually any cells, prokaryotic or eukaryotic, which can be transformed with heterologous DNA or RNA and which can be grown or maintained in culture, may be used in the practice of the invention. Examples include bacterial cells such as E. coli, mammalian cells such as mouse, hamster, pig, goat, primate, etc., and other eukaryotic cells such as Xenopus cells, Drosophila cells, Zebrafish cells, C. elegans cells, and the like. They may be of a wide variety of tissue types, including mast cells, fibroblasts, oocytes and lymphocytes, and they may be primary cells or cell lines. Specific examples include CHO cells and COS cells. Cell-free transcription systems also may be used in lieu of cells.

As used herein, a “vector” may be any of a number of nucleic acids into which a desired sequence may be inserted by restriction and ligation for transport between different genetic environments or for expression in a host cell. Vectors are typically composed of DNA although RNA vectors are also available. Vectors include, but are not limited to, plasmids, phagemids and virus genomes. A cloning vector is one which is able to replicate in a host cell, and which is further characterized by one or more endonuclease restriction sites at which the vector may be cut in a determinable fashion and into which a desired DNA sequence may be ligated such that the new recombinant vector retains its ability to replicate in the host cell. In the case of plasmids, replication of the desired sequence may occur many times as the plasmid increases in copy number within the host bacterium or just a single time per host before the host reproduces by mitosis. In the case of phage, replication may occur actively during a lytic phase or passively during a lysogenic phase.

An expression vector is one into which a desired DNA sequence may be inserted by restriction and ligation such that it is operably joined to regulatory sequences and may be expressed as an RNA transcript. Vectors may further contain one or more marker sequences (i.e., reporter sequences) suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art (e.g., beta-galactosidase or alkaline phosphatase), and genes which visibly affect the phenotype of transformed or transfected cells, hosts, colonies or plaques. Preferred vectors are those capable of autonomous replication and expression of the structural gene products present in the DNA segments to which they are operably joined.

As used herein, a marker or coding sequence and regulatory sequences are said to be “operably” joined when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript might be translated into the desired protein or polypeptide.

The precise nature of the regulatory sequences needed for gene expression may vary between species or cell types, but shall in general include, as necessary, 5′ non-transcribed and 5′ non-translated sequences involved with the initiation of transcription and translation respectively, such as a TATA box, capping sequence, CCAAT sequence, and the like. Especially, such 5′ non-transcribed regulatory sequences will include a promoter region which includes a promoter sequence for transcriptional control of the operably joined coding sequence. Regulatory sequences may also include enhancer sequences or upstream activator sequences as desired. The vectors of the invention may optionally include 5′ leader or signal sequences. The choice and design of an appropriate vector is within the ability and discretion of one of ordinary skill in the art.

Expression vectors containing all the necessary elements for expression are commercially available and known to those skilled in the art. See, e.g., Sambrook et al., Molecular Cloning: A Laboratory Manual, Second Edition, Cold Spring Harbor Laboratory Press, 1989. Cells are genetically engineered by the introduction into the cells of heterologous nucleic acid, usually DNA, molecules, encoding a lipoic acid ligase mutant. The heterologous nucleic acid molecules are placed under operable control of transcriptional elements to permit the expression of the heterologous nucleic acid molecules in the host cell.

Preferred systems for mRNA expression in mammalian cells are those such as pcDNA3.1 (available from Invitrogen, Carlsbad, Calif.) that contain a selectable marker such as a gene that confers G418 resistance (which facilitates the selection of stably transfected cell lines) and the human cytomegalovirus (CMV) enhancer-promoter sequences. Additionally, suitable for expression in primate or canine cell lines is the pCEP4 vector (Invitrogen, Carlsbad, Calif.), which contains an Epstein Barr virus (EBV) origin of replication, facilitating the maintenance of plasmid as a multicopy extrachromosomal element. Another expression vector is the pEF-BOS plasmid containing the promoter of polypeptide Elongation Factor lcc, which stimulates efficiently transcription in vitro. The plasmid is described by Mishizuma and Nagata (Nuc. Acids Res. 18:5322, 1990), and its use in transfection experiments is disclosed by, for example, Demoulin (Mol. Cell. Biol. 16:4710-4716, 1996). Still another preferred expression vector is an adenovirus, described by Stratford-Perricaudet, which is defective for E1 and E3 proteins (J. Clin. Invest. 90:626-630, 1992). The use of the adenovirus as an Adeno.P1A recombinant is disclosed by Warnier et al., in intradermal injection in mice for immunization against PIA (Int. J. Cancer, 67:303-310, 1996).

The invention also embraces so-called expression kits, which allow the artisan to prepare a desired expression vector or vectors. Such expression kits include at least separate portions of each of the previously discussed coding sequences. Other components may be added, as desired, as long as the previously mentioned sequences, which are required, are included.

It will also be recognized that the invention embraces the use of the above described, lipoic acid ligase mutant encoding nucleic acid containing expression vectors, to transfect host cells and cell lines, be these prokaryotic (e.g., E. coli), or eukaryotic (e.g., rodent cells such as CHO cells, primate cells such as COS cells, Drosophila cells, Zebrafish cells, Xenopus cells, C. elegans cells, yeast expression systems and recombinant baculovirus expression in insect cells). Especially useful are mammalian cells such as human, mouse, hamster, pig, goat, primate, etc., from a wide variety of tissue types including primary cells and established cell lines.

Various methods of the invention also require expression of fusion proteins in vivo. The fusion proteins are generally recombinantly produced proteins that comprise the lipoic acid ligase acceptor peptides. Such fusions can be made from virtually any protein and those of ordinary skill in the art will be familiar with such methods. Further conjugation methodology is also provided in U.S. Pat. Nos. 5,932,433; 5,874,239 and 5,723,584.

In some instances, it may be desirable to place the lipoic acid ligase or mutant thereof and possibly the fusion protein under the control of an inducible promoter. An inducible promoter is one that is active in the presence (or absence) of a particular moiety. Accordingly, it is not constitutively active. Examples of inducible promoters are known in the art and include the tetracycline responsive promoters and regulatory sequences such as tetracycline-inducible T7 promoter system, and hypoxia inducible systems (Hu et al. Mol Cell Biol. 2003 December; 23(24):9361-74). Other mechanisms for controlling expression from a particular locus include the use of synthetic short interfering RNAs (siRNAs).

As used herein with respect to nucleic acids, the term “isolated” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art.

As used herein, a subject shall mean an organism such as an insect, a yeast cell, a worm, a fish, or a human or animal including but not limited to a dog, cat, horse, cow, pig, sheep, goat, chicken, rodent e.g., rats and mice, primate, e.g., monkey. Subjects include vertebrate and invertebrate species. Subjects can be house pets (e.g., dogs, cats, fish, etc.), agricultural stock animals (e.g., cows, horses, pigs, chickens, etc.), laboratory animals (e.g., mice, rats, rabbits, etc.), zoo animals (e.g., lions, giraffes, etc.), but are not so limited. Methods of the invention may be used to introduce labels for MRI, PET, or multiphoton imaging, etc. into and for detection in live animals. Methods of the invention may be applied to living animals, for example, transgenic animals, thus subjects of the invention may be transgenic animals.

The compositions, as described above, are administered in effective amounts for labeling of the target proteins. The effective amount will depend upon the mode of administration, the location of the cells being targeted, the amount of target protein present and the level of labeling desired.

The methods of the invention, generally speaking, may be practiced using any mode of administration that is medically acceptable, meaning any mode that produces effective levels of the active compounds without causing clinically unacceptable adverse effects. A variety of administration routes are available including but not limited to oral, rectal, topical, nasal, intradermal, or parenteral routes. The term “parenteral” includes subcutaneous, intravenous, intramuscular, or infusion.

When peptides are used, in certain embodiments one desirable route of administration is by pulmonary aerosol. Techniques for preparing aerosol delivery systems containing peptides are well known to those of skill in the art. Generally, such systems should utilize components which will not significantly impair the biological properties of the peptides or proteins (see, for example, Sciarra and Cutie, “Aerosols,” in Remington's Pharmaceutical Sciences, 18th edition, 1990, pp 1694-1712; incorporated by reference). Those of skill in the art can readily determine the various parameters and conditions for producing protein or peptide aerosols without resort to undue experimentation.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Lower doses will result from other forms of administration, such as intravenous administration. In the event that a response in a subject is insufficient at the initial doses applied, higher doses (or effectively higher doses by a different, more localized delivery route) may be employed to the extent that subject tolerance permits. Multiple doses per day are contemplated to achieve appropriate systemic levels of compounds.

The agents may be combined, optionally, with a pharmaceutically-acceptable carrier.

The invention in other aspects includes pharmaceutical compositions. When administered, the pharmaceutical preparations of the invention are applied in pharmaceutically-acceptable amounts and in pharmaceutically-acceptably compositions. Such preparations may routinely contain salt, buffering agents, preservatives, compatible carriers, and the like. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

Various techniques may be employed for introducing nucleic acids of the invention into cells, depending on whether the nucleic acids are introduced in vitro or in vivo in a host. Such techniques include transfection of nucleic acid-CaPO.sub.4 precipitates, transfection of nucleic acids associated with DEAE, transfection with a retrovirus including the nucleic acid of interest, liposome mediated transfection, and the like. For certain uses, it is preferred to target the nucleic acid to particular cells. In such instances, a vehicle used for delivering a nucleic acid of the invention into a cell (e.g., a retrovirus, or other virus; a liposome) can have a targeting molecule attached thereto. For example, a molecule such as an antibody specific for a surface membrane protein on the target cell or a ligand for a receptor on the target cell can be bound to or incorporated within the nucleic acid delivery vehicle. For example, where liposomes are employed to deliver the nucleic acids of the invention, proteins which bind to a surface membrane protein associated with endocytosis may be incorporated into the liposome formulation for targeting and/or to facilitate uptake. Such proteins include capsid proteins or fragments thereof tropic for a particular cell type, antibodies for proteins which undergo internalization in cycling, proteins that target intracellular localization and enhance intracellular half life, and the like. Polymeric delivery systems also have been used successfully to deliver nucleic acids into cells, as is known by those skilled in the art. Such systems even permit oral delivery of nucleic acids.

Other delivery systems can include time-release, delayed release or sustained release delivery systems. Such systems can avoid repeated administrations of the labeling reagents. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono- di- and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which the anti-inflammatory agent is contained in a form within a matrix such as those described in U.S. Pat. Nos. 4,452,775, 4,667,014, 4,748,034 and 5,239,660 and (b) diffusional systems in which an active component permeates at a controlled rate from a polymer such as described in U.S. Pat. Nos. 3,832,253, and 3,854,480.

A preferred delivery system of the invention is a colloidal dispersion system. Colloidal dispersion systems include lipid-based systems including oil-in-water emulsions, micelles, mixed micelles, and liposomes. A preferred colloidal system of the invention is a liposome. Liposomes are artificial membrane vessels which are useful as a delivery vector in vivo or in vitro. It has been shown that large unilamellar vessels (LUV), which range in size from 0.2-4.0 μm can encapsulate large macromolecules. RNA, DNA, and intact virions can be encapsulated within the aqueous interior and be delivered to cells in a biologically active form (Fraley, et al., Trends Biochem. Sci., (1981) 6:77). In order for a liposome to be an efficient gene transfer vector, one or more of the following characteristics should be present: (1) encapsulation of the gene of interest at high efficiency with retention of biological activity; (2) preferential and substantial binding to a target cell in comparison to non-target cells; (3) delivery of the aqueous contents of the vesicle to the target cell cytoplasm at high efficiency; and (4) accurate and effective expression of genetic information.

Liposomes may be targeted to a particular tissue by coupling the liposome to a specific ligand such as a monoclonal antibody, sugar, glycolipid, or protein. Liposomes are commercially available from Gibco BRL, for example, as LIPOFECTIN™ and LIPOFECTACE™, which are formed of cationic lipids such as N-[1-(2, 3 dioleyloxy)-propyl]-N,N,N-trimethylammonium chloride (DOTMA) and dimethyl dioctadecylammonium bromide (DDAB). Methods for making liposomes are well known in the art and have been described in many publications. Liposomes also have been reviewed by Gregoriadis, G. in Trends in Biotechnology, (1985) 3:235-241.

In one important embodiment, the preferred vehicle is a biocompatible microparticle or implant that is suitable for implantation into the mammalian recipient. Exemplary bioerodible implants that are useful in accordance with this method are described in PCT International application no. PCT/US/03307 (Publication No. WO 95/24929, entitled “Polymeric Gene Delivery System”). PCT/US/03307 describes a biocompatible, preferably biodegradable polymeric matrix for containing an exogenous gene under the control of an appropriate promoter. The polymeric matrix is used to achieve sustained release of the exogenous gene in the patient. In accordance with the instant invention, the fugetactic agents described herein are encapsulated or dispersed within the biocompatible, preferably biodegradable polymeric matrix disclosed in PCT/US/03307.

The polymeric matrix preferably is in the form of a microparticle such as a microsphere (wherein an agent is dispersed throughout a solid polymeric matrix) or a microcapsule (wherein an agent is stored in the core of a polymeric shell). Other forms of the polymeric matrix for containing an agent include films, coatings, gels, implants, and stents. The size and composition of the polymeric matrix device is selected to result in favorable release kinetics in the tissue into which the matrix is introduced. The size of the polymeric matrix further is selected according to the method of delivery which is to be used. Preferably when an aerosol route is used the polymeric matrix and agent are encompassed in a surfactant vehicle. The polymeric matrix composition can be selected to have both favorable degradation rates and also to be formed of a material which is bioadhesive, to further increase the effectiveness of transfer. The matrix composition also can be selected not to degrade, but rather, to release by diffusion over an extended period of time.

In another exemplary embodiment the delivery system is a biocompatible microsphere that is suitable for local, site-specific delivery. Such microspheres are disclosed in Chickering et al., Biotech. and Bioeng., (1996) 52:96-101 and Mathiowitz et al., Nature, (1997) 386:410-414.

Both non-biodegradable and biodegradable polymeric matrices can be used to deliver the agents of the invention to the subject. Biodegradable matrices are preferred. Such polymers may be natural or synthetic polymers. Synthetic polymers are preferred. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water and further, optionally is cross-linked with multivalent ions or other polymers.

In addition, important embodiments of the invention include pump-based hardware delivery systems, some of which are adapted for implantation. Such implantable pumps include controlled-release microchips. A preferred controlled-release microchip is described in Santini, J T Jr., et al., Nature, 1999, 397:335-338, the contents of which are expressly incorporated herein by reference.

Use of a long-term sustained release implant may be particularly suitable for treatment of chronic conditions. Long-term release, as used herein, means that the implant is constructed and arranged to delivery therapeutic levels of the active ingredient for at least 30 days, and preferably 60 days. Long-term sustained release implants are well-known to those of ordinary skill in the art and include some of the release systems described above.

The invention will be more fully understood by reference to the following examples. These examples, however, are merely intended to illustrate the embodiments of the invention and are not to be construed to limit the scope of the invention.

### Example 1

### Experimental

### General Considerations

All reactions were performed in dry solvents under an inert nitrogen atmosphere and with vigorous stirring unless otherwise stated. Temperatures stated refer to the external medium. Oil baths, in combination with IKA heating mantles and thermocouples, were used to achieve elevated temperatures. Ice baths were used to cool to 0° C. Solid CO₂ in acetone was used to cool to −78° C. TLC was performed on EMD 60 F254 aluminum backed plates and visualized under UV light or stained with KMnO₄ solution. Purification over silica was achieved using EMD 60 (230-400 mesh). All commercial reagents were bought from Sigma-Aldrich, VWR, Fisher Scientific or TCI America with purities of over 95% and, unless otherwise stated, were used as received. Anhydrous solvents were bought from Sigma-Aldrich or VWR and used as received. ¹H and ¹³C NMR were recorded on a Varian 400 MHz spectrometer and processed offline using ACD/NMR Processor Acaderhic Edition using residual isotopic solvent as an internal reference for organic deuterated solvents. The following abbreviations were used during assignment: s=singlet, d=doublet, t=triplet, q=quartet, quint=quintet, m=multiplet, br=broad. Mass spectra were measured by the University of Notre Dame Mass Spectrometry and Proteomics Facility. Non-radioactive HPLC spectra were recorded on a Hitachi LaChrom Elite system with L-2455 diode array detector (Hitachi, Tokyo, Japan) equipped with a Luna C18(2) 100 Å250×4.6 mm 5 micron column (Phenomenex, Torrance, Calif.) using UV detection at 220 nm. Non-radioactive semi-preparative HPLC were performed on the same system equipped with a Luna C18(2), 100 Å250×10.0 mm 10 micron column (Phenomenex, Torrance, Calif.).

[¹⁸F]fluoride ion trapped on an ion exchange ORTG cartridge was purchased from the University of California Radiopharmaceutical Facility. RP-HPLC Purification and analysis of radiolabeled compounds was performed using a Waters 600 system (Waters, Milford, Mass.) with a Shimadzu-10A UV-vis detector (Shimadzu, Koyoto, Japan) and an in line CsI(TI) radiation detector (Carroll & Ramsey, Berkeley, Calif.) also using Phenomenex Luna C18(2) columns. SEC analysis used the same system equipped with a Phenomenex BioSep SEC S3000 300×7.8 mm column. C₁₈light and Oasis HLB sep-paks were purchased from Waters (Milford, Mass.). NuPAGE SDS-PAGE buffer was purchased from Thermo Fisher Scientific Life Technologies (Waltham, Mass.) and used after the addition of 2.5% v:v mercaptoethanol. Ni-NTA spin columns (0.2 mL resin volume) were purchased from Thermo Fisher Scientific Life Technologies (Waltham, Mass.). Mouse serum was purchased from Sigma-Aldrich (St. Louis, Mo.).

The LAP (Ac-GFEIDKVWYDLDA-OH, 95% purity) and scrambled LAP (Ac-EFDDWKYADVGLI, 95% purity) peptides, both acetylated at the N-terminus, were custom synthesized by Wuxi AppTech Co. Ltd. (Hong Kong).

### Synthetic Chemistry

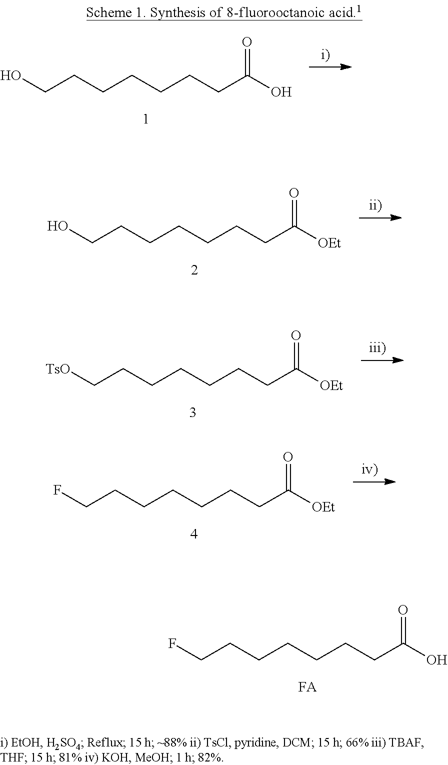

Ethyl 8-hydroxyoctanoate (2): 8-hydroxyoctanoic acid (950 mg, 5.93 mmol) was dissolved in ethanol (80 mL). H₂SO₄ (800 μL) was added and the resulting solution was heated at reflux for 15 h and then allowed to cool to ambient temperature. The reaction was then diluted with Et₂O (50 mL) and washed with saturated NaHCO_{3 (aq)}(3×50 mL). The aqueous extracts were combined and washed with Et₂O (1×50 mL). The combined organic extracts were washed with brine (1×50 mL), dried (MgSO₄), filtered and concentrated to give the crude ethyl ester 2 (980 mg) which was used without further purification in the next step.

Ethyl 8-[[(4-methylphenyl)sulfonyl]oxy]octanoate (3): Crude 2 (955 mg, 5.07 mmol) was dissolved in DCM (50 mL) in an inert nitrogen atmosphere. Pyridine (817 μL, 10.1 mmol) and tosyl chloride (1.16 g, 6.08 mmol) were added and the reaction was stirred at ambient temperature for 15 h. TLC analysis revealed incomplete conversion of 3, hence another portion of tosyl chloride (1.16 g, 6.08 mmol) was added and the reaction was stirred for a further 15 h. The reaction was diluted with DCM (50 mL) and extracted with 1 M HCl (3×50 mL). The aqueous extracts were washed with DCM (1×50 mL) and the combined organic extracts were washed with brine (1×50 mL), dried (MgSO₄), filtered and concentrated to give the crude product which was purified over silica (6:1-5:1 hexane:ethyl acetate) to give 3 (1.15 g, 3.36 mmol, 66%). ¹H NMR (400 MHz, CDCl₃): δ_{H}=1.20-1.35 (m, 9H), 1.55-1.70 (m, 4H), 2.27 (t, J=7.5 Hz, 2H), 2.46 (s, 3H), 4.02 (t, J=6.5 Hz, 2H), 4.13 (q, J=7 Hz, 2H), 7.36 (d, J=8.5 Hz, 2H), 7.80 (d, J=8.5 Hz, 2H). Matches data previously reported.¹

Ethyl 8-fluorooctanoate (4): 3 (974 mg, 2.84 mmol) was dissolved in THF (60 mL) in an inert nitrogen atmosphere. TBAF (11.4 mL of 1M solution in THF, 11.4 mmol) was added and the resulting solution was stirred at ambient temperature overnight. The reaction was then concentrated and purified over silica (8:1 hexane:.ethyl acetate) to give 4 (436 mg, 2.29 mmol, 81%). ¹H NMR (400 MHz, CDCl₃): δ_{H}=1.26 (t, J=7.0 Hz, 3H), 1.30-1.45 (m, 4H), 1.60-1.75 (m, 4H), 2.30 (t, J=7.5 Hz, 2H), 4.13 (q, J=7.0 Hz, 2H), 4.44 (dt, J=6.0, 47.0 Hz, 2H). Matches data previously reported.¹

8-Fluorooctanoic Acid (FA): 4 (436 mg, 2.29 mmol) was dissolved in a mixture of MeOH and 5 N KOH (16 mL, 1:1 v:v) and the resulting solution was stirred at ambient temperature for 1 h. The reaction was acidified with 1 M HCl and extracted with EtOAc (3×25 mL). The combined organic extracts were dried (MgSO₄), filtered and concentrated to give the crude product which was purified by recrystallization from pet. ether to give FA (303 mg, 1.87 mmol, 82%). ¹H NMR (400 MHz, CDCl₃): 1.35-1.50 (m, 6H), 1.60-1.75 (m, 4H), 2.37 (t, J=7.5 Hz, 2H), 4.44 (dt, J=6.5, 47.5 Hz, 2H). Matches data previously reported.

Production of Lp1A: Lp1A was expressed and purified in Escherichia coli BL21 (DE3) Gold (Stratagene) as described previously. The plasmid pYFJ16 for the expression of Lp1A- His₆ was a kind gift from John Cronan (University of Illinois). Briefly, transformed cells were grown in 1 L of lysogeny broth containing 100 μg/mL ampicillin at 37° C. and 250 rpm to an OD600 of 0.8. The enzyme expression was induced with 1 mM Isopropyl β-_{D}-1-thiogalactopyranoside (IPTG) and the induced cultures were grown for 3 h at 30° C. The bacteria were harvested by centrifugation and resuspended in lysis buffer (50 mM Tris base, 300 mM NaCl, pH 7.8) containing protease inhibitor cocktail (complete EDTA-free, Roche). The cells were lysed by sonication and the cell debris was removed by centrifugation at 4° C. for 20 min at 18,000 g. Lp1A-His₆ was purified by affinity chromatography using Ni²⁺-NTA agarose resin (QIAGEN) using a standard protocol recommended by the manufacturer. Pure Lp1A was dialyzed overnight at 4° C. against PBS buffer pH 7.4 and its purity was confirmed by SDS-PAGE. Lp1A was stored at 80° C. for up to 6 months with no measurable loss in activity.

Production of 2G10-Fab-LAP: Anti-human uPAR 2G10 Fab was previously discovered from a human naïve B cell Fab phage-displayed library. For the generation of the of 2G10-LAP Fab, the sequence for the LAP peptide was inserted at the C-terminus of the heavy chain using standard cloning methods. Fabs were expressed and purified in Escherichia coli BL21 (DE3) Gold (Stratagene). Cultures were grown in 1 L of 2×yeast extract and tryptone containing 100 μg/mL ampicillin and 0.1% glucose at 37° C. and 200 rpm to an OD600 of 0.6. The protein expression was induced with the addition of 1 mM and grow overnight at 20° C. The cells were harvested by centrifugation and the cell pellet was resuspended in ice-cold 1×TES (0.2 M Tris pH 8, 0.5 mM EDTA, 0.5 M sucrose). The cell suspension was mixed with the same volume of ice cold ddH₂O and incubated on ice for 30 min. The solution was then pelleted and the supernatant (periplasmic fraction) was used for the purification. The periplasmic fraction was applied to a column containing 1 mL of Ni-NTA beads prewashed with wash buffer 1 (50 mM Tris pH 8, 250 mM NaCl). The column was washed with 20 column volumes of wash buffer 2 (50 mM Tris pH 8, 500 mM NaCl, 20 mM Imidazole) and the Fab was eluted with 1 column volume of elution buffer (50 mM Tris pH 8, 500 mM NaCl, 500 mM Imidazole). The purified Fab was dialyzed overnight at 4° C. against against PBS buffer pH 7.4 and analyzed by SDS-PAGE.

Production of Lp1AΔHis: For the generation of Lp1A without His₆-tag, the specific cleavage site of the Tobacco Etch Virus (TEV) protease, ENLYFQG, was inserted between the His₆-tag and the coding sequence of Lp1A using standard cloning methods. Lp1AΔHis was expressed and purified using the same protocol described for Lp1A. His₆-tagged TEV protease was added to the purified enzyme in a ratio of 1:100 (w/w) and was dialyzed overnight at 4° C. against PBS buffer pH 7.4. The His₆-tagged TEV protease was removed by affinity chromatography using Ni²⁺-NTA agarose resin. Lp1AΔHis was stored at 80° C. for up to 6 months with no measurable loss in activity.

Labeling of LAP peptide with non-radioactive FA: The following stock solutions were generated in PBS: LAP peptide (600 μM); FA (7.5 mM); Lp1A (˜40-100 μM); ATP and Mg(OAc)₂ (30 mM and 50 mM respectively). Each reagent was diluted to the appropriate final concentration in PBS and the resulting solution was incubated at 30° C. At specified time-points, 100 μL aliquots were withdrawn and diluted with 100 μL of 360 mM EDTA. 99 μL, of this solution was analyzed via RP-HPLC using a 20 minute 30-60% gradient of MeCN in H₂O (plus 0.1% TFA). In order to prepare a sample of FA-labeled LAP for ESI-MS analysis, a 1 mL labeling reaction was purified via SP-HPLC using the same gradient. ESI-MS (m/z): Calculated (C₈₄H₁₁₈N₁₅O₂₅F.H⁺) 1756.92; Found 1756.85.

Synthesis of [¹⁸F]-FA: K[¹⁸F] (100-500 mCi) was eluted off an ORTG cartridge using 0.5 mL of a K_{2.2.2}/K₂CO₃ solution (12.6 mg/mL K_{2.2.2}, 2 mg/mL K₂CO₃, 9:1 v:v MeCN:H₂O). The resulting solution was subjected to 3×drying cycles at 110° C. Tosylate 1 (−2 mg) was dissolved in anhydrous MeCN (300 μL) and added to the dried [¹⁸F] mixture and the resulting solution was sealed and heated at 90° C. for 10 minutes. It was then dilute to ˜5 mL with H₂O and purified via semi-preparative RP-HPLC (60-90% gradient of MeCN in H₂O plus 0.1% TFA; product eluted at ˜17 mins). Purified 2 was diluted to ˜30 mL with H₂O and loaded onto a C₁₈-light sep-pak. The sep-pak was washed with H₂O (10 mL) and then the activity was eluted in 5 N KOH (1 mL). The resulting solution was heated at 90° C. for 10 minutes and then cooled for 1-2 mins over ice. The solution was neutralized with acetic acid (750 μL), diluted to ˜30 mL with H₂O and [¹⁸F]-FA was loaded onto an Oasis HLB sep-pak. The sep-pak was then washed with H₂O (10 mL) and [¹⁸F]-FA was then eluted in MeCN (2 mL). This solution was then concentrated for 1 h at 50° C. under reduced pressure and [¹⁸F]-FA was then dissolved in PBS for use in subsequent radiolabeling studies. RP-HPLC analysis of [¹⁸F]-FA used 45:65:0.1 v:v:v MeCN:H₂O:TFA as the eluent (1 mL/min).

Radiolabeling of LAP Peptide and 2G10-Fab-LAP: The stock solutions of LAP peptide or 2G10-Fab-LAP, Lp1A or Lp1AΔHis and ATP/Mg(OAc)₂ were diluted to the appropriate concentration in PBS (200 μL final volume). [¹⁸F]-FA (0.2-3 mCi) in PBS was added and the resulting solution was incubated at 30° C. for 10-15 mins. EDTA (360 mM, 200μL) was added to quench Lp1A activity. An aliquot (15 μL) was withdrawn from the quenched reaction and added to SDS-PAGE reducing buffer and then heated at 95° C. for 5 minutes. This solution was then analyzed via radio-TLC (eluent: 7:3:0.1 EtOAc:hexanes:acetic acid). RP-HPLC analysis of reaction mixtures used 45:65:0.1 v:v:v MeCN:H₂O:TFA as the eluent (1 ml/min). SEC analysis used aqueous solutions of 100 mM sodium phosphate (pH 6.8) and 300 mM NaCl (2 mL/min).

Radiolabeling of 2G10-Fab-LAP with [¹⁸F]-SFB: A solution of 2G10-Fab-LAP (10 μM) and [¹⁸F]-SFB (˜200 μCi) in 50 mM sodium borate buffer (pH=8.5) was heated at 40° C. for 10 minutes. The reaction mixture was then analyzed via radio-TLC (eluent: 7:3:0.1 EtOAc:hexanes:acetic acid) to determine the conjugation yield.

Purification of [¹⁸F]-2G10-Fab-LAP: The reaction solution was diluted with 100 mM imidazole in PBS to a final concentration of 10 mM imidazole. The solution was then loaded onto a nickel-affinity spin column. The column was washed 3 times with 25 mM imidazole before elution with 3 times with 250 mM imidazole. 2G10-Fab-[¹⁸F]-LAP was present in each of the three elutions, but was most concentrated in the first one. The concentration of 2G10-Fab in each sample was determined using the BCA protein assay kit (Thermo Fisher Scientific Life Technologies, Waltham, Mass.) following 5-fold dilution with PBS.

Serum Stability Studies: 2G10-Fab-[¹⁸F]-LAP (˜400 μCi) was added to mouse serum (1 mL) and incubated at 37° C. for 1 h. MeCN (1 mL) was added and the resulting suspension was centrifuged at 2000 rpm for 5 minutes. ˜1 mL of the supernatent was filtered through a 0.45 μM filter and the resulting solution was analyzed via SEC and radio-TLC using conditions previously described (vide supra).

Measurement of 2G10-Fab-FA-LAP Affinity for uPAR: Stocks solutions were diluted to the following concentrations in 100 μL PBS: 2G10-Fab-LAP (10 μM), Lp1AΔHis (50 μM), FA (750 μM), ATP (3 mM), Mg(OAc)₂ (5 mM). The resulting solution was incubated at 30° C. for 15 h and then quenched with 100 μL. 360 mM EDTA. Kinetic constants this sample, along with 2G10 and 2G10-Fab-LAP, were determined using an Octet RED384 instrument (ForteBio). Four concentrations of each Fab (500 nM, 250 nM, 100 nM and 50 nM) were tested for binding to the biotinylated antigen (human uPAR) immobilized on ForteBio streptavidin SA biosensors. All measurements were performed at room temperature in 384-well microplates and the running buffer was PBS with 0.1% (w/v) bovine serum albumin (BSA) and 0.02% (v/v) Tween 20. Biotinylated human uPAR was loaded for 180 s from a solution of 150 nM, baseline was equilibrated for 60 s, and then the Fabs were associated for 120 s followed by 300 s disassociation. Between each Fab sample, the biosensor surfaces were regenerated three times by exposing them to 10 mM glycine, pH 1.5 for 5 s followed by PBS for 5 s. Data were analyzed using a 1:1 interaction model on the ForteBio data analysis software 8.2.

### Results

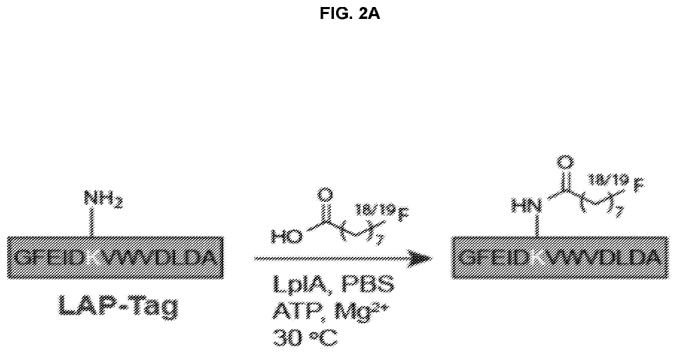

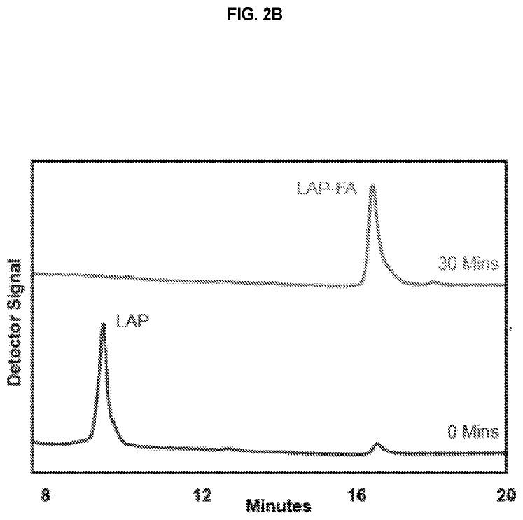

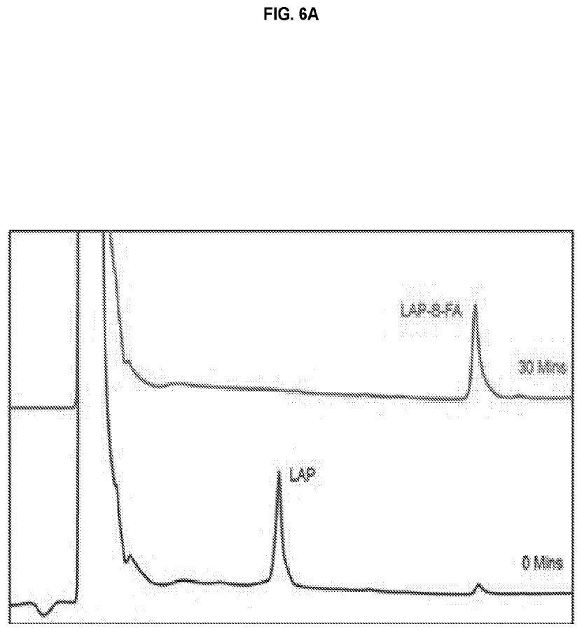

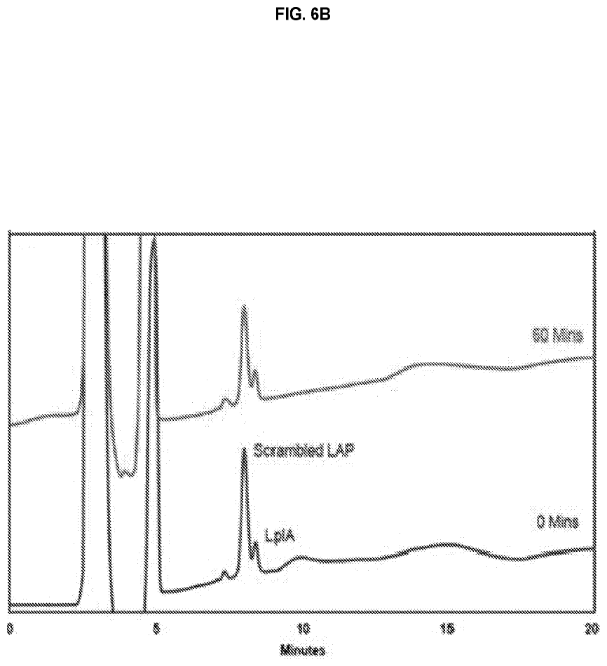

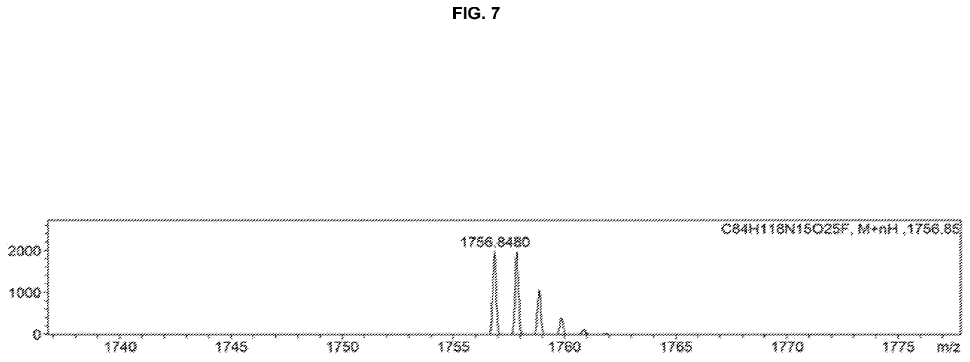

Initially, we sought to confirm that non-radioactive [¹⁹F]-FA is a viable substrate for Lp1A. This compound was synthesized in 4 steps from 8-hydroxyoctanoic acid as previously reported (Nagatsugi, et al., J. Nucl. Med. 2014, 48(2):304). We then incubated the LAP peptide (60 μM) with Lp1A (500 nM) and FA (750 μM) at 30° C. in PBS along with the required enzymatic co-factors ATP (3 mM) and Mg²⁺(5 mM). At various time points aliquots were withdrawn, Lp1A activity quenched with EDTA, and reaction progress measured via RP-HPLC (FIG. 2). Conversion of LAP to a more hydrophobic species, consistent with conjugation to [¹⁹F]-FA, was complete at 30 minutes. The product peak was isolated via semi-preparative RP-HPLC and confirmed as [¹⁹F]-FA-LAP by ESI-MS (m/z found=1756.85; expected=1756.92; (FIG. 6A and FIG. 6B). As an initial assessment of the site-specificity of Lp1A, we also incubated a ‘scrambled’ LAP peptide (EFDDWKYADVGLI) with the same reaction components. No productive reaction was observed after 60 minutes, suggesting that only the precise amino acid sequence of the LAP-tag is recognized by Lp1A.

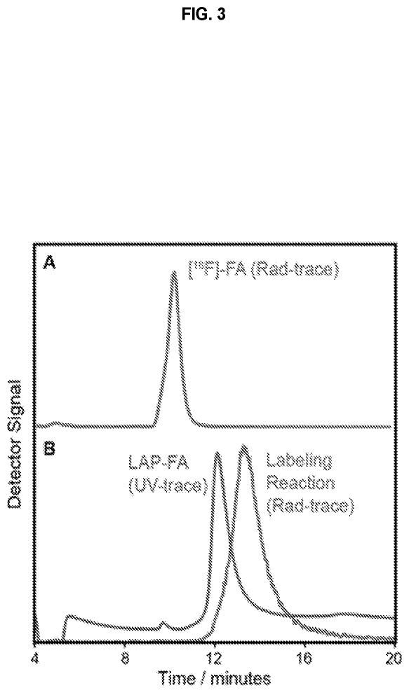

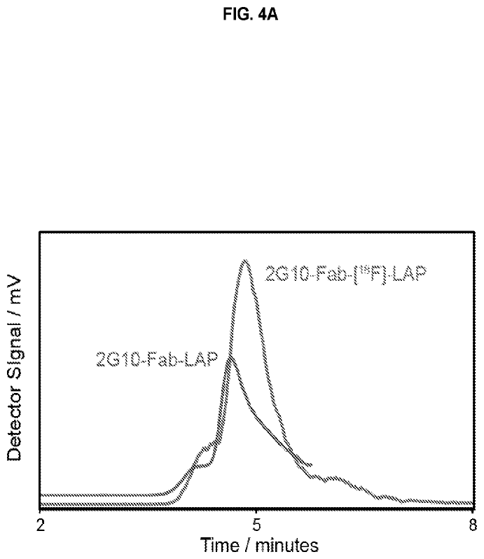

We next synthesized [¹⁸F]-FA in 2 steps from tosylate 1, making minor changes to a previously published protocol (Scheme 1) (Nagatsugi, et al., Nucl. Med. Biol. 1994, 21(6):809). Briefly, 1 was radiofluorinated under standard conditions and the resulting alkyl [¹⁸F]-fluoride 2 was purified by semi-preparative RP-HPLC. The ethyl ester was then hydrolyzed in 5 N KOH and [¹⁸F]-FA was immobilized on a reversed-phase sep-pak, washed to remove all traces of KOH, and eluted in MeCN. In order to remove all MeCN prior to dissolving [¹⁸F]-FA in PBS for radiolabeling, this solution was heated under reduced pressure at 50° C. for 1 h. Higher temperatures led to a significant loss of activity, presumably due to the volatility of [¹⁸F]-FA. The total time from production of [¹⁸F] to dissolving [¹⁸F]-FA in PBS ready for peptide/protein radiofluorination was ˜180 mins. The non-decay corrected yield for the radiosynthesis is 8±1.5% (average of 4 separate syntheses), allowing us to generate ˜40 mCi of [¹⁸F]-FA from ˜500 mCi of [¹⁸F], sufficient to investigate the radiofluorination of the LAP peptide and subsequently 2G10-Fab-LAP. [¹⁸F]-FA was ˜98% pure by RP-HPLC (FIG. 3A) with no evidence of any impurities in the UV-trace.

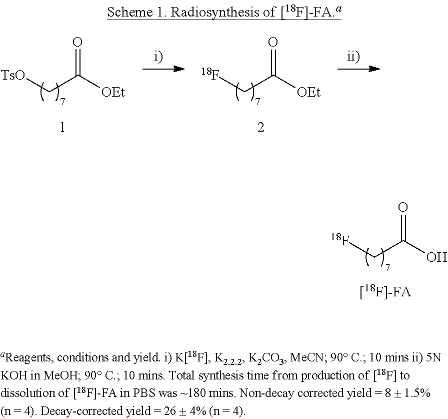

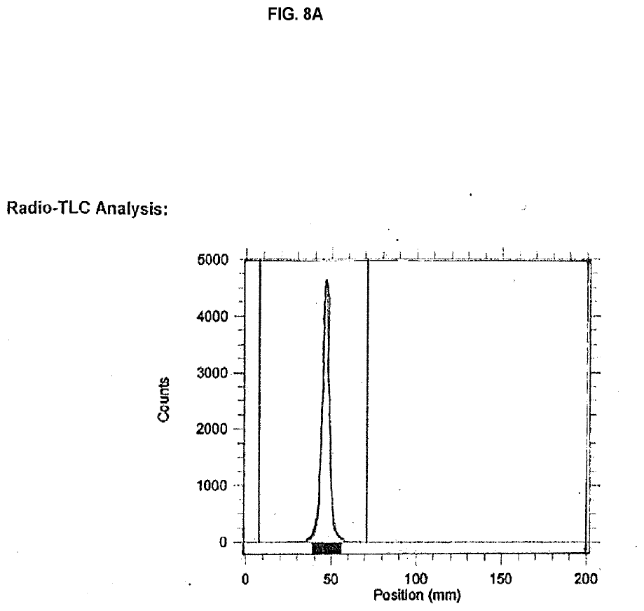

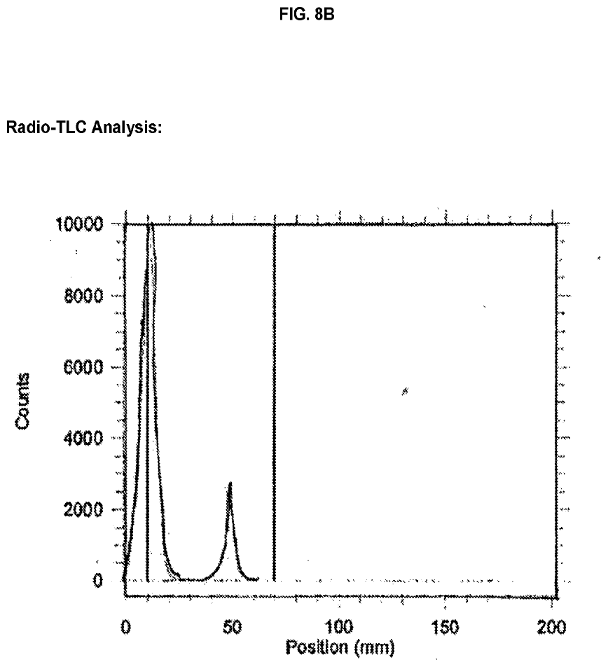

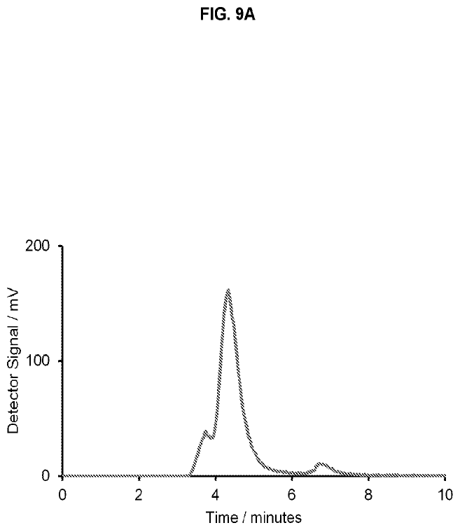

The prosthetic concentrations typical of radiofluorination are far lower than we had tested previously with [¹⁹F]-FA (1-10 μM), hence we first investigated whether labeling was still rapid and high yielding in these conditions. Approximately 200 μCi of [¹⁸F]-FA was added to a 200 μL solution of the LAP peptide (60 μM, 12 nmol) and Lp1A (5 μM) and the consumption of [¹⁸F]-FA was measured by radio-TLC following quenching with EDTA (see FIG. 8A and FIG. 8B for representative examples of radio-TLC analyses). After just 10 minutes, ˜90% of the prosthetic had been consumed and converted to a more polar species which remained on the baseline of the TLC plate, consistent with the conjugation of [¹⁸F]-FA to the LAP peptide: The formation of LAP-[¹⁸F]-FA was confirmed by RP-HPLC and comparison to [¹⁹F]-FA-LAP (FIG. 3B). Interestingly, a control reaction containing Lp1A. (10 μM) but no LAP peptide exhibited a 30% consumption of [¹⁸F]-FA by radio-TLC, suggestive of productive peptide/protein labeling. We reasoned that [¹⁸F]-FA might bind non-covalently to Lp1A, generating a false-positive signal in our TLC assay. This was confirmed by diluting a sample of this reaction with reducing SDS-PAGE buffer and briefly heating it to 95° C. to break any non-covalent bonds, after which radio-TLC analysis measured no consumption of [¹⁸F]-FA. Pleasingly, the conjugation yields measured in the presence of the LAP peptide were unchanged after this treatment due to the covalent bond formed under these conditions. Moving forward, we treated a small sample (˜1-2 μL) of each reaction in this way to ensure our radio-TLC analyses were accurately reflecting productive bioconjugation.

TABLE 1
LplA Conjugates [¹⁸F]-FA to the LAP Peptide.^{a}

| | [Peptide]/ | [LplA]/ | |
|---|---|---|---|
| Peptide | μM | μM | Yield/%^{b} |
| LAP | 60 | 5 | 91 ± 1.5 (n = 3) |
| LAP | 60 | 0 | 0 |
| Sc. | 60 | 5 | 0 |
| LAP | | | |
| — | 0 | 10 | 0 |

^{a}General considerations: All reactions performed in 200 μL PBS + 3 mM ATP + 5 mM Mg(OAc)₂ at 30° C. for 10 mins and quenched with EDTA (180 mM final concentration) prior to analysis.
^{b}Radio-TLC yields measured after treatment of reaction sample with SDS-PAGE buffer at 95° C. for 5 mins. 7:3:0.1 EtOAc:Hexane:Acetic acid used as eluent.

We then explored the lower limits of peptide concentration at which high conjugation yields (>80%) were retained. Keeping [Lp1A] fixed at 5 μM, we incrementally reduced [LAP] and discovered that yields dropped below 15 μM (Table 2). Raising [Lp1A] to 10 μM restored labeling down to a [LAP] of 5 μM. Reducing [LAP] still further lowered the yields, which could not be improved by adding more Lp1A (up to 50 μM).

TABLE 2
Establishing Lower Concentration of LAP at
which Radioconjugation Yields are >80%^{a}

| [LAP]/ | [LplA]/ | Average | Range of |
|---|---|---|---|
| μM | μM | Yield/%^{b} | Yields/%^{b} |
| 60 | 5 | 91 ± 1.5 | 90-93 |
| | | (n = 3) | |
| 15 | 5 | 92 | — |
| 5 | 5 | 57 | — |
| 5 | 10 | 83 ± 10.8 | 67-93 |
| | | (n = 4) | |
| 2.5 | 50 | 53 | — |
| 1 | 50 | 28 | — |

^{a}General considerations: All reactions performed in 200 μL PBS + 3 mM ATP + 5 mM Mg(OAc)₂ at 30° C. for 10 mins and quenched with EDTA (180 mM final concentration) prior to analysis.
^{b}Radio-TLC yields measured after treatment of reaction sample with SDS-PAGE buffer at 95° C. for 5 mins. 7:3:0.1 EtOAc:Hexane:Acetic acid used as eluent.

Encouraged by the rapid and high yielding labeling of the isolated LAP-tag, we moved onto radiofluorinating 2G10-Fab. The LAP-tag was inserted at the C-terminus of the heavy chain using standard cloning methods. This position was chosen as we had already inserted His₆-tags here without reducing epitope affinity. The resulting construct, 2G10-Fab-LAP, was expressed in E. coli BL21 (DE3) and purified via nickel affinity chromatography. We then use radio-TLC analysis to measure conjugation of [¹⁸F]-FA to 2G10-Fab-LAP (Table 3). The optimal conditions identified for the LAP peptide (5 μM 2G10-LAP, 10 μM Lp1A) gave inconsistent conjugation yields of 49-83%. Doubling the concentration of 2G10-LAP (10 μM, 2 nmol) and slightly extending the reaction time to 15 minutes gave reliably high conjugation yields (92±7%, n=4). Pleasingly, 2G10 without a LAP-tag was barely radiofluorinated (3±1%, n=3) under identical conditions, illustrating the site-specificity of the methodology. Following a standard protocol (Cai, et al., J. Nucl. Med. 2014, 48(2):304), we also measured the radiolabeling of 2G10-Fab-LAP (10 μM) with [¹⁸F]-SFB after a 10 minute incubation at 40° C. Radio-TLC measured significantly lower conjugation yields of 22±1.2% (n=3), highlighting the benefits of our enzymatic approach.

TABLE 3
Radiofluorination of 2G10-Fab-LAP^{a}

| | | | Reaction | |
|---|---|---|---|---|
| | [Protein]/ | [LplA]/ | Time/ | |
| Protein | μM | μM | mins | Yield/%^{b} |
| 2G10-LAP | 5 | 10 | 10 | 49-83 |
| 2G10-LAP | 10 | 10 | 15 | 92 ± 7 (n = 4) |
| 2G10 | 10 | 10 | 15 | 3 ± 1 (n = 3) |

^{a}General considerations: All reactions performed in 200 μL PBS + 3 mM ATP + 5 mM Mg(OAc)₂ at 30° C. for 10 mins and quenched with EDTA (180 mM final concentration) prior to analysis.
^{b}Radio-TLC yields measured after treatment of reaction sample with SDS-PAGE buffer at 95° C. for 5 mins. 7:3:0.1 EtOAc:Hexane:Acetic acid used as eluent.

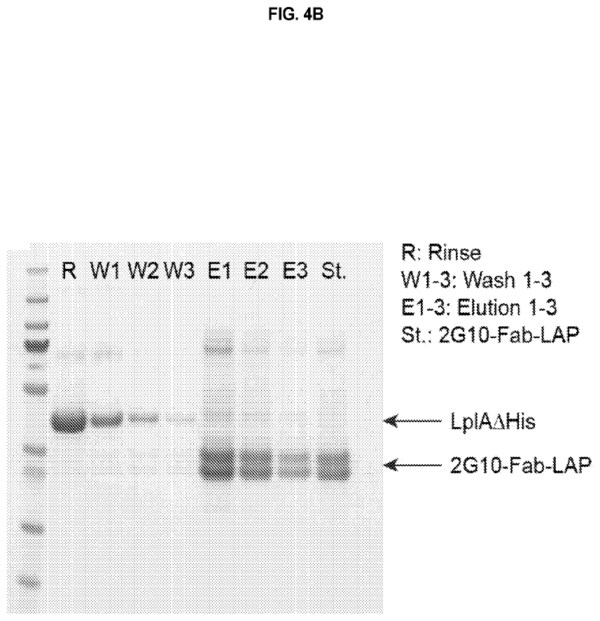

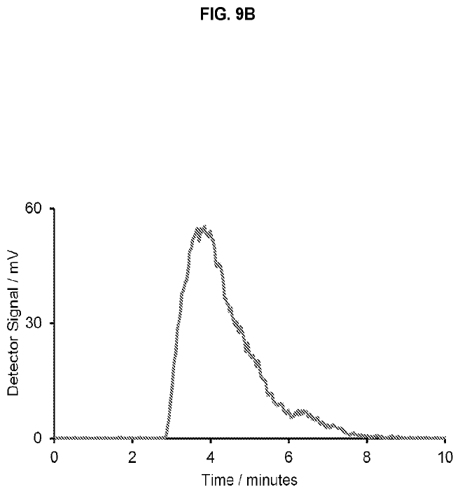

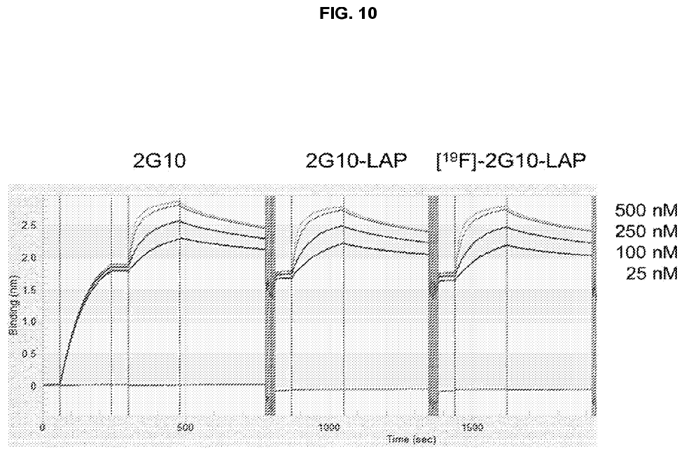

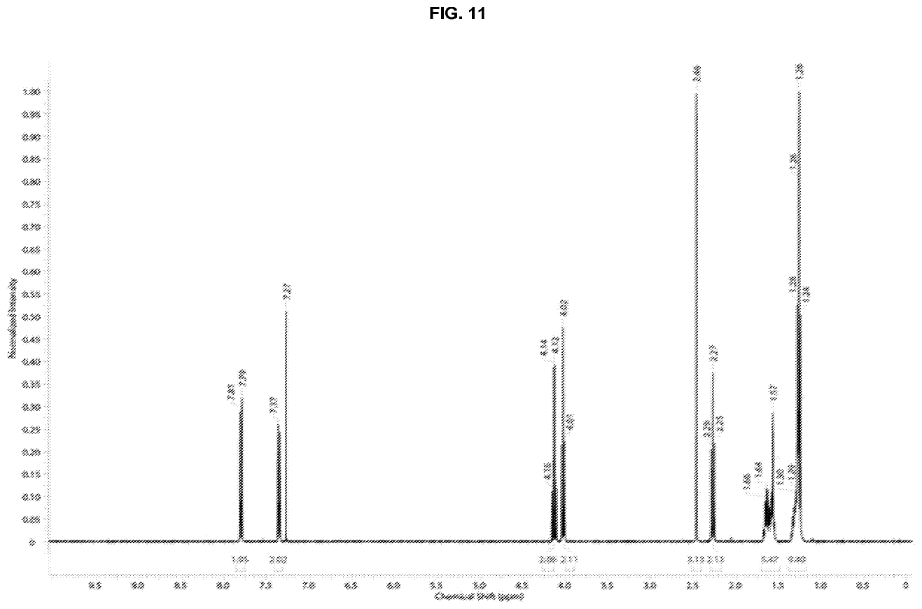

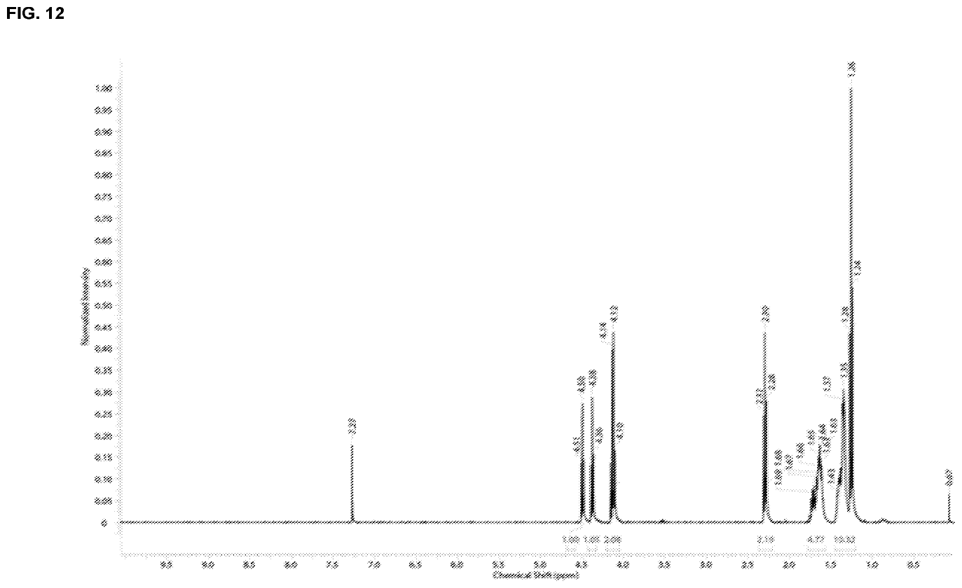

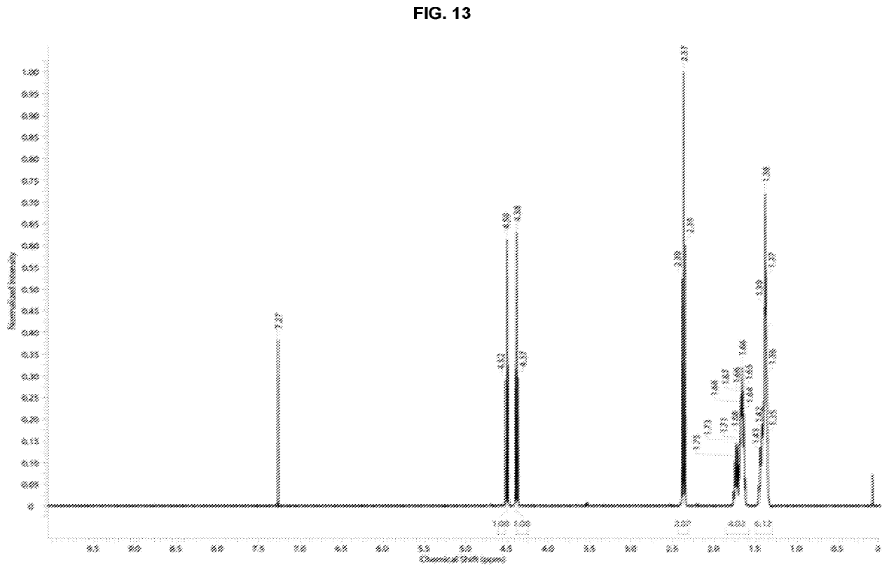

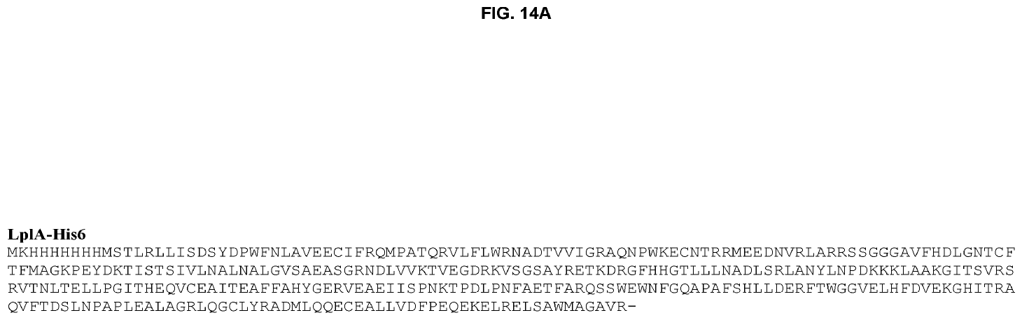

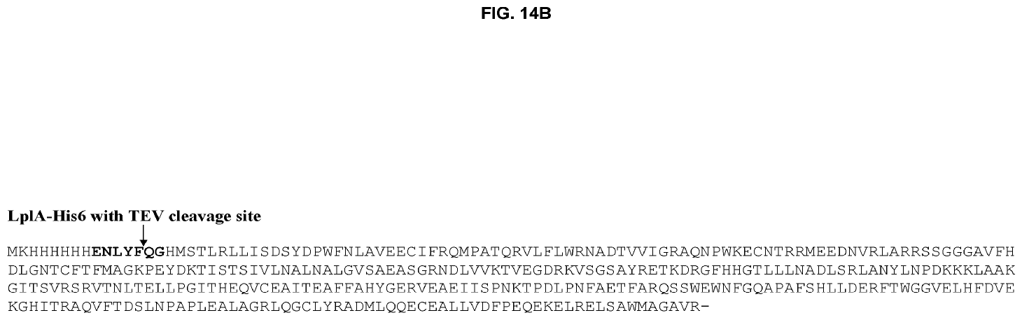

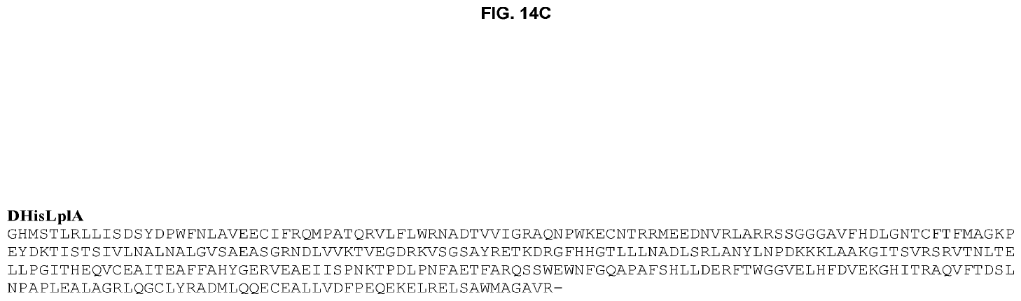

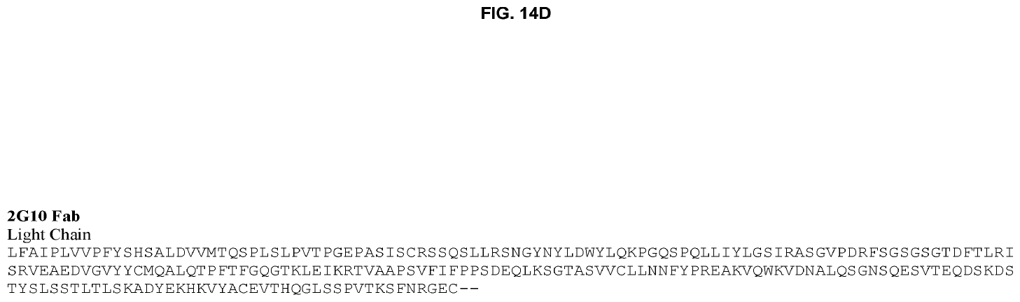

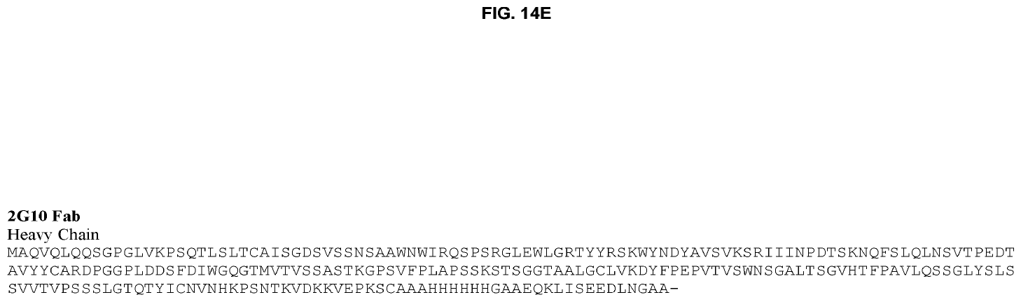

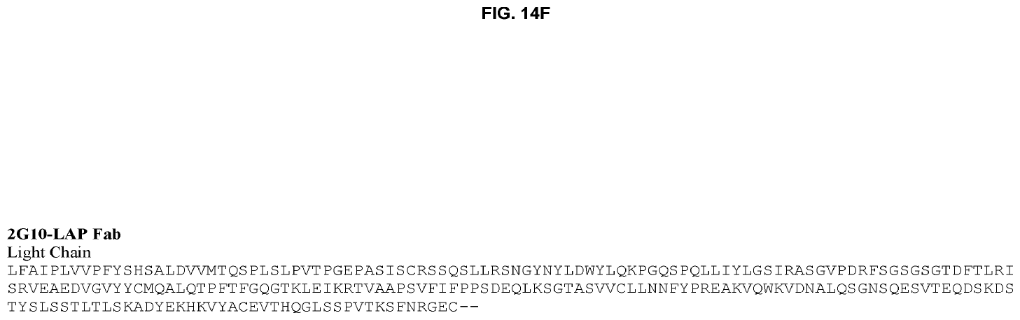

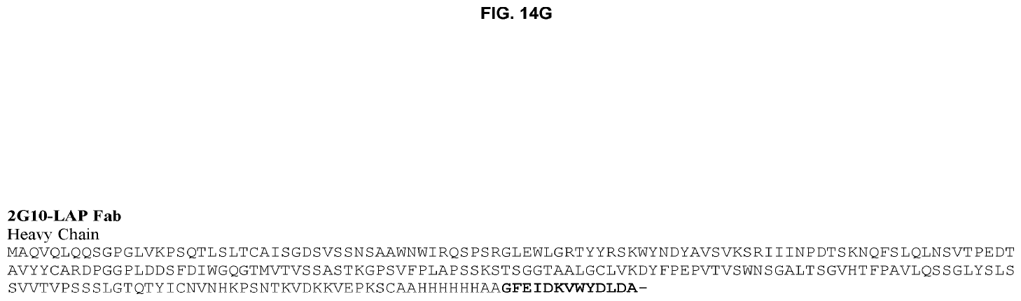

Having demonstrated efficient radiolabeling, we then developed a rapid purification scheme to deliver high specific activity [¹⁸F]-2G10-Fab-LAP for animal studies. In our hands, separating [¹⁸F]-2G10-Fab-LAP from Lp1A by size-exclusion chromatography was not possible. We next attempted to bind 2G10-[¹⁸F]-LAP to a protein L spin column; however no retention of activity was observed after 10 minutes incubation at room temperature. We then inserted a Myc epitope tag at the C-terminus of the heavy chain, immediately following the LAP-tag. Unfortunately, we could not purify 2G10-[¹⁸F]-LAP with anti-Myc beads within our stringent 10 minute time window. Our previous experience with 2G10-Fab-LAP informed us that we could use its His₆-tag for rapid purification, however to do so we needed to remove the His₆-tag from Lp1A. To achieve this, we inserted a TEV protease cleavage site between Lp1A and its His₆-tag. Once Lp1A had been isolated from E. coli, but prior to any radiochemistry, we incubated it with TEV overnight at 4° C. to remove the His₆-tag. The radiofluorination performance of the resulting enzyme, Lp1AΔHis, was indistinguishable from the wild type enzyme. Following radiofluorination, [¹⁸F]-2G10-Fab-LAP bound to nickel beads within the desired 10 minute incubation period. Residual Lp1AΔs and [¹⁸F]-FA was washed off the column and the purified probe was subsequently eluted in PBS+250 mM imidazole. We confirmed radiotracer purity by SEC and SDS-PAGE (FIG. 4). To assess the serum stability of 2G10-Fab-[¹⁸F]-LAP, we incubated it in mouse serum for 1 h and analyzed the resulting radioactivity by SEC (FIG. 9). No release of low molecular weight material consistent with cleavage of [¹⁸F]-FA from the protein in serum was observed, suggesting [¹⁸F]-2G10-Fab-LAP is sufficiently stable for use in vivo.

After repeating the bioconjugation chemistry with an excess of [¹⁹F]-FA, to ensure labeling of all 2G10-Fab-LAP present in the sample, we measured the impact of the prosthetic on 2G10's affinity for uPAR (Table 4). No significant differences in binding affinity were observed between 2G10-Fab, 2G10-Fab-LAP and [¹⁹F]-2G10-Fab-LAP.

TABLE 4
Dissociation Constants, Measured using Octet
Instrument, for 2G10-Fabs with uPAR.

| Protein | K_{D} (nM) |
|---|---|
| 2G10-Fab | 38 ± 2.7 |
| 2G10-Fab-LAP | 31 ± 2.2 |
| 2G10-Fab-[¹⁹F]-LAP^{a} | 31 ± 2.5 |

^{a}Labeling conditions: 2G10-Fab-LAP (10 μM), LplAΔHis (50 μM), FA (750 μM), ATP (3 mM), Mg(OAc)₂ (5 mM). The resulting solution was incubated at 30° C. for 15 h and then quenched with 100 μL 360 mM EDTA.

To this point we had used small quantities for [¹⁸F]-FA (˜200 μCi) for the radiochemical optimization studies. To establish that the methodology can prepare enough radiotracer for an animal imaging study, we executed the radiolabeling of 2G10-Fab-LAP with 2-3 mCi⁻of [¹⁸F]-FA. Initially, radio-TLC reported disappointing yields of 27-38% (Table 5). Increasing both the amount of 2G10-Fab-LAP (10 nmol) and Lp1A restored high conjugation yields (95±7%, n=4). In summary, starting with 2.78-3.02 mCi of [¹⁸F]-FA we isolated 1.19-1.62 mCi of [¹⁸F]-2G10-Fab-LAP following purification (69±12% decay-corrected yield). Analysis of the purified sample using the BCA assay demonstrated ˜100% recovery of 2G10-Fab-LAP (˜10 nmol protein). The total conjugation process, including purification, lasted 55-60 mins and the specific activity of the generated radiotracer was 119-162 Ci/mmol.

TABLE 5
Optimization of 2G10-Fab-LAP Radiofluorination
using 2-3 mCi of [¹⁸F]-FA.

| [2G10- | | Reaction | Amount of | |
|---|---|---|---|---|
| Fab-LAP]/ | [LplA]/ | Volume/ | [¹⁸F]-FA/ | |
| μM | μM | μL | mCi | Yield^{b} |
| 10 | 10 | 200 | 0.35-0.55 | 92 ± 7 |
| 10 | 10 | 200 | 2.0-2.3 | 27-38 |
| 10 | 50 | 200 | 2.7-2.8 | 19 ± 3 |
| 25 | 50 | 400 | 2.7 | 95 ± 7 |

^{a}General considerations: All reactions performed in PBS + 3 mM ATP + 5 mM Mg(OAc)₂ at 30° C. for 15 mins and quenched with EDTA (180 mM final concentration) prior to analysis.
^{b}Radio-TLC yields measured after treatment of reaction sample with SDS-PAGE buffer at 95° C. for 5 mins. 7:3:0.1 EtOAc:Hexane:Acetic acid used as eluent.

### Discussion

The present examples show that the bacterial enzyme Lp1A recognizes the unnatural substrate [¹⁸F]-FA and can couple it rapidly and selectively to a known acceptor peptide (LAP-tag). The biochemistry was highly efficient on both the isolated LAP-tag and a LAP-tagged recombinant Fab (2G10-Fab-LAP), with conjugation yields of >80% attained after short 10-15 minute incubations. We also established a rapid purification scheme, enabling isolation of [¹⁸F]-2G10-Fab-LAP in mCi quantities within 1 hour from the start of bioconjugation. A schematic summary of our methodology is shown in FIG. 5. A distinguishing feature of this methodology is its efficacy with low amounts of protein substrate (2-10 nmol). In comparison, yields measured with the current gold standard in the field, [¹⁸F]-SFB, were significantly lower (22%). Our results are comparable to those reported for radiofluorination using a tetrazine ligation, one of the most efficient bioconjugation reactions known.

The enzymatic radiofluorination proceeds in aqueous conditions at neutral pH and near ambient temperature, mild conditions likely to preserve the structural integrity of delicate biomolecules. The selectivity shown by Lp1A for the LAP-tag exerts control over the labeling site, again preventing a loss of biological activity during labeling. We measured complete retention of 2G10 affinity for its cognate receptor (uPAR) following labeling, highlighting these beneficial features. Additionally, [¹⁸F]-2G10-Fab-LAP was very stable in mouse serum, another essential attribute for successful imaging.

Being a close structural analogue of a known Lp1A substrate, [¹⁸F]-FA was a logical choice for proof of concept. Based on our experience with this molecule, we are now actively working to refine its structure to reduce synthesis time and volatility. Because Lp1A is tolerant of structural variation in its substrates, we are optimistic that we can improve upon [¹⁸F]-FA without impairing its biochemistry. Despite the lengthy, low-yielding synthesis of [¹⁸F]-FA, and hence the likely low specific activity of this prosthetic, the efficiency of our methodology generated [¹⁸F]-2G10-Fab-LAP with specific activities similar to those reported previously for other bioconjugation techniques (Flavell, et al., J. Am. Chem. Soc. 2008, 130(28):9106; Glaser, et al., J. Nucl. Med. 2013, 54(11):1981; Cai, et al., J. Nucl. Med. 2014, 48(2):304). We are confident that a refined [¹⁸F]-prosthetic with a streamlined, higher-yielding synthesis will result higher specific activity radiotracers.

Two other groups have developed enzymatic radiofluorination schemes. Rashidian et al. used a sortase to conjugate a tetrazine moiety to an antibody fragment, which enabled radiolabeling with an [¹⁸F]-FDG-trans-cyclooctene prosthetic. They reported yields of 90% using 6 nmol of protein, comparable to our data. Thompson, et al. used a fluorinase enzyme to radiofluorinate a nucleotide coupled to a RGD peptide, again achieving excellent yields albeit with more peptide precursor (˜80 nmol). A direct comparison between our methodology and theirs is difficult as they have yet to report radiofluorination data for higher molecular weight proteins. Moving forward, we are excited to more systematically study the strengths and weaknesses of these exciting enzymatic radiofluorination strategies relative to our own.

### Conclusions

We have developed an enzymatic radiofluorination which uses Lp1A to directly conjugate a [¹⁸F]-prosthetic site-specifically to a protein. Our methodology has several advantages compared to traditional chemical [¹⁸F]-bioconjugations. The labeling is rapid and high yielding under mild, aqueous conditions and with minimal amounts of protein substrate (1-10 nmol). The mild conditions and site-specificity preserve the epitope affinity of delicate proteins. In addition, the serum stability of the construct and the ability to scale to mCi amounts suggest animal and human imaging is feasible.

### Example 2

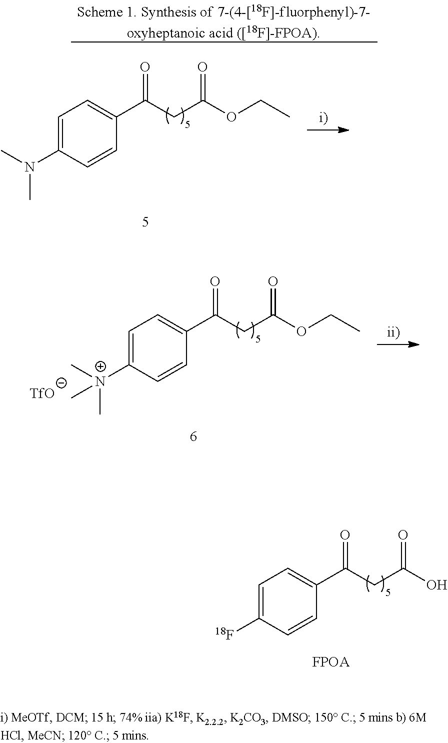

5 and non-radioactive [¹⁹F]-FPOA was purchased from Rieke Metals (Lincoln, Nebr.).

Ethyl 7-[4-(N,N,N-trimethylamino)phenyl]-7-oxyheptanoate triflate (6): 5 (950 mg, 3.26 mmol) was dissolved in anhydrous DCM (25 mL) and the resultant solution was stirred at room temperature overnight. The solution was then concentrated under reduce pressure and the crude product loaded onto a short silica plug which was washed with 1:1 EtOAc:hexane and then the crude product was eluted with 10% MeOH in DCM. This solution was concentrated and 6 was recrystallized from EtOH/Et₂O as an off-white solid (1.1 g, 2.41 mmol, 74%). ¹H NMR (CDCl₃, 400 MHz): δ_{H}=8.16 (d, J=9 Hz, 2H), 7.98 (d, J=9 Hz, 2H), 4.13 (q, J=7 Hz, 2H), 3.80 (s, 9H), 3.00 (t, J=7 Hz, 2H), 2.33 (t, J=7.5 Hz, 2H), 1.60-1.85 (m, 4H), 1.43 (m, 2H), 1.26 (t, J=7 Hz, 3H).

7-(4-[¹⁸F]-Fluorophenyl)-7-oxyheptanoic acid: K[¹⁸F] (100-500 mCi) was eluted off an ORTG cartridge using 0.7 mL of a K_{2.2.2}/K₂CO₃ solution (12.6 mg/mL K_{2.2.2}, 2 mg/mL K₂CO₃, 9:1 v:v MeCN:H₂O). The resulting solution was subjected to 3×drying cycles at 110° C. 6 (˜6 mg) was dissolved in anhydrous DMSO (300 μL) and added to the dried [¹⁸F] mixture and the resulting solution was sealed and heated at 150° C. for 5 minutes. The resulting solution was cooled over ice for 1 minute and then diluted with 25 mL H₂O and loaded onto a HLB-plus sep-pak (Waters, Milford, Mass.). The sep-pak was washed with H₂O (10 mL) and a mixture of MeCN:H₂O (3:7, v:v, 3 mL) before the activity was eluted through a MCX sep-pak (Waters, Milford, Mass.) with 2% formic acid in MeCN (2 mL). 6 M HCl (1 mL) was added and the reaction was sealed and heated to 120 Tosylate 1 (˜2 mg) was dissolved in anhydrous MeCN (300 μL) and added to the dried [¹⁸F] mixture and the resulting solution was sealed and heated at 120° C. for 5 minutes. The resulting solution was cooled over ice for 1 minute, diluted with 25 mL H₂O and loaded onto a HLB-plus sep-pak. The sep-pak was washed with H₂O (10 mL) and [¹⁸F]-FPOA was eluted with MeCN (2 mL). The solution was concentrated at 110° C. under reduced pressure for 10 mins before the activity was re-dissoloved in PBS +10% DMSO prior to use for peptide/protein radiolabeling. The identity of [¹⁸F]-FPOA was confirmed by analytical RP-HPLC (eluent=1:1:0.01 MeCN:H₂O:TFA) and comparison with a non-radioactive sample of FPOA.

Radiolabeling of LAP peptide and 2G10-Fab-LAP: [¹⁸F]-FPOA and ^{W371}Lp1AΔHis were used in place of [¹⁸F]-FA and Lp1AΔHis under labeling conditions identical to those reported for [¹⁸F]-FA/Lp1AΔHis.

The present invention provides, inter alia, novel radiolabeled prosthetics, methods of conjugating the prosthetics in to a polypeptide conjugate and methods of using the polypeptide conjugates in diagnostic imaging modalities and various additional analyses and processes. While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined not with reference to the above description, but instead should be determined with reference to the appended claims along with their full scope of equivalents.

All publications and patent documents cited in this application are incorporated by reference in their entirety for all purposes to the same extent as if each individual publication or patent document were so individually denoted. By their citation of various references in this document, Applicants do not admit any particular reference is “prior art” to their invention.

1. A method of conjugating a detectable label or therapeutic agent to a protein fusion between a targeting polypeptide and an acceptor polypeptide for a lipoic acid prosthesis comprising a domain recognized by a lipoic acid ligase and said detectable label or said therapeutic agent, said method comprising:
contacting said fusion with said prosthetic and said lipoic acid ligase under conditions such that said lipoic acid ligase transfers said prosthetic to said acceptor polypeptide, thereby conjugating said detectable label of therapeutic agent to said protein fusion .

2. The method according to claim 1, wherein said prosthetic comprises a detectable label which is a radioisotope.

3. The method according to claim 2, wherein said radioisotope is a positron emitting radioisotope.

4. The method according to claim 3, wherein said radioisotope is ¹⁸F.

5. The method according to claim 1, wherein said acceptor polypeptide is at least about 95% homologous with the sequence GFEIDKVWYDLDA (SEQ. ID. NO: 2) .

6. The method according to claim 1, wherein said lipoic acid ligase is of a sequence at least about 95% homologous with the sequence of SEQ. ID. NO: 1.

7. The method according to claim 1, wherein said prosthetic is of a structure according to Formula I: 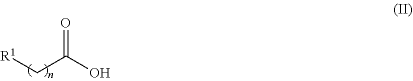
in which R¹ is a detectable label selected from a radioisotope, a fluorophore or a mass spectrometric label, or R¹ is a reactive functional group, a therapeutic agent, e.g., a toxin or, optionally, R¹ is substituted or unsubstituted alkyl, substituted or unsubstituted heteroalkyl, substituted or unsubstituted aryl, or substituted or unsubstituted heteroaryl, functionalized with said detectable moiety or said therapeutic moiety; the index n is an integer from 6-18.

8. The method according to claim 7, wherein said prosthetic is:
F—(CH₂)₇—COOH.

9. A protein conjugate formed by a method according to claim 1.

10. The protein conjugate according to claim 9, in formulation with a pharmaceutically acceptable carrier.

11. A method of acquiring a positron emission tomographic image, said method comprising:
(a) administering to a subject in need of obtaining a positron emission tomographic image a diagnostically useful amount of a protein conjugate according to claim 9; and
(b) acquiring said positron emission tomographic image of said subject following said administering.

12. A kit for preparing a conjugate by a method according to claim 1, said kit comprising:
(a) a vessel containing said prosthetic;
(b) a vessel containing said lipoid acid ligase;
(c) a vessel containing said protein fusion; and, optionally one or more solvents, buffers, devices for administering said conjugate to a subject, and instructions for preparing said conjugate and/or instructions for using said conjugate to obtain a positron emission tomographic image of a subject in need thereof.
